(19) United States
(12) Patent Application Publication
(10) Pub. No.: US 2016/0258938 A1
(43) Pub. Date: Sep. 8, 2016

(54) METHOD OF DETECTING AN ANALYTE IN A SAMPLE

(71) Applicant: Headway Technologies, Inc., Milpitas, CA (US)

(72) Inventors: Jaime E. Arenas, San Ramon, CA (US); Hetian Gao, Fremont, CA (US); Bin Guo, Pleasanton, CA (US); Celine Hu, Tiburon, CA (US); Koki Kawamura, Chiba (JP)

(21) Appl. No.: 15/061,554

(22) Filed: Mar. 4, 2016

Related U.S. Application Data
(60) Provisional application No. 62/128,416, filed on Mar. 4, 2015.

(51) Int. Cl. G01N 33/53

(52) U.S. Cl. CPC G01N 33/5306; G01N 33/5308

(57) ABSTRACT

Methods of detecting a target analyte in a sample are provided. Aspects of the method include: (a) contacting the sample with (i) a first capture agent that specifically binds the target analyte and (ii) a reporter complex under conditions sufficient to produce a sandwich complex; (b) separating the sandwich complex from the sample; and (c) releasing a detectable tag from the sandwich complex. The reporter complex may include a first specific binding member linked to a second capture agent that specifically binds the target analyte, and a second specific binding member specifically bound to the first specific binding member. In some cases, the second specific binding member is linked to a detectable tag. The releasing step may be achieved using a displacement binding member that is complementary to the first or second specific binding member. Also provided are compositions, systems and kits for practicing the subject methods.

### CROSS-REFERENCE

This application claims priority benefit of U.S. Provisional Application No. 62/128,416, filed Mar. 4, 2015, which application is incorporated herein by reference in its entirety and for all purposes.

### INTRODUCTION

Highly sensitive methods for the detection and quantitation of specific analytes in fluids are valuable in life sciences, diagnostics and pharmaceutical industries. For example, the detection of analytes such as toxins, microorganisms, and other disease biomarkers at low concentrations affords early diagnosis and increases the success rate of medical treatments. Disease biomarkers include proteins and nucleic acids that may be analyzed in clinical samples such as plasma, serum, urine and saliva. One strategy to detect analytes present at concentrations below the lower limit of detection of available technologies is to pre-concentrate the analyte present in a larger sample down to a concentration and volume suitable for detection. This can be accomplished by selectively binding the analyte to a solid support before directly measuring the bound analyte. Alternatively, the bound analyte can be released from the solid support before it is measured. However, methods utilized to release bound analytes from a solid support often use harsh conditions that are not compatible with biological molecules or downstream detection methods.

### SUMMARY OF THE INVENTION

Methods of detecting a target analyte in a sample are provided. Aspects of the method include: (a) contacting the sample with (i) a first capture agent that specifically binds the target analyte and (ii) a reporter complex under conditions sufficient to produce a sandwich complex; (b) separating the sandwich complex from the sample; and (c) releasing a detectable tag from the sandwich complex. The reporter complex may include a first specific binding member linked to a second capture agent that specifically binds the target analyte, and a second specific binding member specifically bound to the first specific binding member. In some cases, the second specific binding member is linked to a detectable tag. The releasing step may be achieved using a displacement binding member that is complementary to the first or second specific binding member. Also provided are compositions, systems and kits for practicing the subject methods.

### BRIEF DESCRIPTION OF THE DRAWINGS

The disclosure is best understood from the following detailed description when read in conjunction with the accompanying drawings. It is emphasized that, according to common practice, the various features of the drawings are not necessarily to-scale. The dimensions of the various features are arbitrarily expanded or reduced for clarity.

FIG. 1 provides a schematic depicting three components of one embodiment of the subject methods. (A) The first component includes a first agent “A” with specific binding affinity for a target analyte “T” (not shown) where “A” is optionally tethered to a solid support “S”. (B) The second component is reporter complex “R” which includes a second agent “B” with specific binding affinity for the target analyte, and a displaceable complex “C”, which further includes a first specific binding member (e.g., a first nucleic acid strand (I)) which is linked to “B”, and a second specific binding member (e.g., a second nucleic acid strand (II)) which is at least partially complementary to the sequence of I and bound to it by hybridization of complementary sequences. Furthermore, “II” has properties that can be directly measured, or it is linked to a detectable tag “M” which is directly or indirectly detectable. (C) The third component is a displacement binding member (e.g., a third nucleic acid) “D” which has complementarity to at least a portion of either I or II, and such complementarity overlaps at least in part the complementarity region between I and II.

FIG. 2 provides a schematic depicting an embodiment of the subject methods of analysis of a sample containing a target analyte of interest. “T” is contacted with reagent R (e.g., as described in FIG. 1) and the affinity agent “A” which can be optionally tethered to a solid surface S. After a suitable incubation period, T is bound to A and B in a sandwich complex A:T:B. Subsequently: 1) the complex is separated from the liquid phase and any unbound reagents are washed off; 2) sandwich complex A:T:B is contacted with displacement binding member “D” which binds to I by hybridization of complementary sequences causing displacement of II; and 3) II or M that is released are measured directly or indirectly using any convenient methods.

FIG. 3 provides a schematic depicting an embodiment of the subject methods of analysis of a sample containing a target analyte of interest similar to FIG. 2. Displacement binding member “D” binds to II by hybridization of complementary sequences causing displacement of II in the form of a complex with D, where II or M are then measured directly or indirectly.

FIG. 4 provides a schematic depicting an embodiment of the subject methods of analysis of a sample containing a target analyte. T is present in a large sample volume (e.g., between 0.1 and 10 ml), and the washed complex A:T:B is exposed to reagent D in a smaller volume, preferably between 0.005 to 0.050 ml, causing II to be displaced into the smaller volume of liquid and to be substantially more concentrated than T was in the original sample.

FIG. 5 provides a schematic depicting a reporter complex of the subject methods that includes multiple copies of first and second binding members (e.g., strand “II”). Thus, when a single type of displacement binding member “D” is added during the subject methods, multiple copies of “II” are released per each A:T:B sandwich complex. Each second binding member may be linked to or may comprise one or more detectable tags “M”.

FIG. 6 provides a schematic depicting a multiplexed method, where second specific binding member (e.g., strand “II”) has a unique addressable tag “G” tethered to it, and multiple versions of the reporter complex “R” are utilized such that there is a correspondence between the specific target binding agent “B” and a unique addressable tag “G”. For example, R1 contains B1 and G1; R2 contains B2 and G2, R3 contains B3 and G3, and specifically recognize target analytes T1, T2, and T3 respectively. In this embodiment, multiple target analytes can be simultaneously detected from the same sample by contacting the sample with a mixed reagent R1-R2-R3 and corresponding binding agents A1, A2, A3 to allow formation of complexes A1:T1:B1, A2:T2:B2, A3:T3:B3. After washing unbound sample components and adding a single universal displacement binding member “D”, corresponding versions of strand “II” with a tethered measurable moiety “M” and a unique addressable tag G1, G2, or G3 are released into the liquid phase. The amount of each target analyte can be simultaneously quantified by measuring the measurable moiety “M” associated with each addressable tag “G” using liquid or solid array formats, where the elements of the array contain corresponding capture agents G1′, G2′ and G3′ to specifically recognize the G1, G2, and G3 tags respectively.

FIG. 7 provides a schematic depicting an embodiment of the subject methods where the detectable tag is released using enzymatic cleavage. The partially double stranded complex between hybrid is designed to contain a site that is cleavable by an enzyme (such as a restriction enzyme, RNAse H, a specific or non-specific RNAse, a double strand specific nuclease or a single strand specific nuclease) to release the detectable moiety M into the liquid phase. With some minor modifications of the hybrid design the assay can be used with other types of nucleases to release M. For example, a) a double strand specific nuclease would digest only the dsDNA region and release M; b) if M is attached to the double stranded region, then a single strand specific nuclease can be used to release M; or c) a general nuclease can be used in any case to release M. In this case, if it is desired that the released M remains attached to a small piece of nucleic acid, this nucleic acid portion can be a derivative that is resistant to nuclease attack such as PNA or LNA; d) If T is RNA and B is DNA, then M can be released with RNAse H or any other RNAse such as RNAseA, T1, T2, U1, U2 etc.

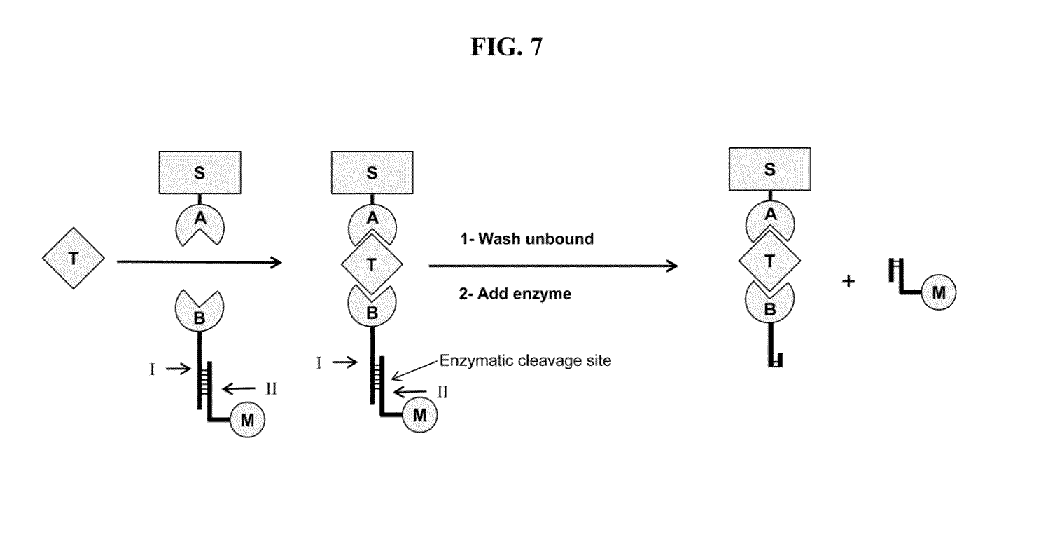

FIG. 8 provides a schematic depicting a model assay performed in Example 1 including the capture of T and subsequent release and detection of a biotin (M) containing oligonucleotide (II) using oligonucleotide D and a streptavidin-alkaline phosphatase conjugate (SAAP).

FIG. 9 provides a schematic depicting embodiments of the subject methods where the detectable tag M is released via several different dissociation points of the complex.

FIG. 10 provides assay results demonstrating a reduction in limit of detection (LOD) when the analyte is captured from a 25 μL or 250 μL sample and subsequently released from a reporter complex into a 10 μL volume.

### DEFINITIONS

Before describing exemplary embodiments in greater detail, the following definitions are set forth to illustrate and define the meaning and scope of the terms used in the description.

As used herein, the term “sample” relates to a material or mixture of materials, in some cases in liquid form, containing or suspected of containing one or more analytes of interest. In some embodiments, the term refers to any plant, animal, fungal, or bacterial (or other microorganism) material containing cells, cellular metabolites, biomarkers, or other analytes of interest, such as, for example, tissue or fluid isolated from an individual (including without limitation plasma, serum, urine, cerebrospinal fluid, lymph, tears, saliva and tissue sections) or from in vitro cell culture constituents, as well as samples from the environment. A sample as described herein may or may not contain cells or cellular material. The term “sample” may also refer to a “biological sample”. As used herein, the term “biological sample” refers to a whole organism or a subset of its tissues, cells or component parts (e.g., body fluids, including, but not limited to, blood, mucus, lymphatic fluid, synovial fluid, cerebrospinal fluid, saliva, amniotic fluid, amniotic cord blood, urine, vaginal fluid, semen, tears, serum, plasma, feces, swabs such as those obtained from the mouth, throat, nose, ears, wounds, or ulcers, tissue biopsies such as those obtained from tumors, organs or other body parts, or tissue sections such as those obtained from cadavers, skin, or hair). A “biological sample” can also refer to a homogenate, lysate or extract prepared from a whole organism or a subset of its tissues, cells or component parts, or a fraction or portion thereof, including but not limited to, plasma, serum, spinal fluid, lymph fluid, the external sections of the skin, respiratory, intestinal, and genitourinary tracts, tears, saliva, milk, blood cells, tumors and organs. In certain embodiments, the sample has been removed from an animal or plant. Biological samples may include cells. The term “cells” is used in its conventional sense to refer to the basic structural unit of living organisms, both eukaryotic and prokaryotic, having at least a cell membrane. In certain embodiments, cells include prokaryotic cells, such as from bacteria. In other embodiments, cells include eukaryotic cells, such as cells obtained from biological samples from animals, plants or fungi. Biological samples may include pathogens such as viruses. In some embodiments, the sample is a biological sample susceptible to infection by a pathogen, such as a virus. The term sample may refer to a water sample, such as an agricultural water, pond, water reservoir, or wastewater sample, or a consumables sample, such as a sample of a food, beverage, cosmetic, etc. In some cases, the water sample can be analyzed for detection, identification, and monitoring of pathogenic and indigenous microorganisms in natural and engineered ecosystems and microcosms such as in municipal waste water purification systems and water reservoirs or in polluted areas undergoing bioremediation. In certain cases, the consumables sample is one containing or suspected of containing an analyte implicated in an allergy (e.g., an allergen implicated in a gluten allergy, dairy allergy, fish allergy, nut allergy, soy allergy, or cosmetic allergy, etc.), or a pathogenic microorganism. In certain instances, the water or consumables sample is one containing or suspected of containing a pharmaceutical agent or product.

As used herein, the terms “determining,” “measuring,” “assessing,” and “assaying” are used interchangeably and include both quantitative and qualitative determinations.

As used herein the terms “affinity agent” and “capture agent” are used interchangeably and refer to an agent that binds an analyte through an interaction that is sufficient to permit the agent to extract the analyte of interest from a mixture of different analytes and/or other sample components. The binding interaction may be mediated by an affinity region of the capture agent. Capture agents may “specifically bind” to one or more analytes. Thus, the term “capture agent” refers to a molecule or a multi-molecular complex which can specifically bind an analyte, e.g., specifically bind an analyte for the capture agent with a dissociation constant (K_{D}) of 10⁻⁶ or less without binding to other targets, such as 10⁻⁶ M or less, 10⁻⁷ M or less, including 10⁻⁸ M or less, e.g., 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, 10⁻¹⁶ M or less, 10⁻¹⁷ M or less, 10⁻¹⁸ M or less, or even less.

The term “capture agent/analyte complex” refers to a complex that results from the specific binding of a capture agent with an analyte, i.e., a “binding partner pair”. The complex may be part of a larger complex (e.g., a sandwich complex). A capture agent and an analyte for the capture agent will typically specifically bind to each other under “conditions suitable for specific binding”, where such conditions are those conditions (in terms of salt concentration, pH, detergent, protein concentration, temperature, etc.) which allow for binding to occur between capture agents and analytes in solution. Such conditions, for example with respect to antibodies and their antigens, are well known in the art (see, e.g., Harlow and Lane (Antibodies: A Laboratory Manual Cold Spring Harbor Laboratory, Cold Spring Harbor, N.Y. (1989)). Conditions suitable for specific binding in some cases permit capture agents and target pairs that have a dissociation constant (K_{D}) of less than about 10⁻⁶ to bind to each other, but not with other capture agents or targets.

As used herein, the terms “affinity” and “avidity” have the same meaning and may be used interchangeably herein. “Affinity” refers to the strength of binding, increased binding affinity being correlated with a lower K_{D}.

Components of interest in a sample are termed “analytes” herein. In some embodiments, the sample is a complex sample containing at least 10², 5×10², 10³, 5×10³, 10⁴, 5×10⁴, 10⁵, 5×10⁵, 10⁶, 5×10⁶, 10⁷, 5×10⁷, 10⁸, 10⁹, 10¹⁰, 10¹¹, 10¹² or more species of analyte. In certain embodiments, the sample is a sample containing 100 or fewer analytes, such as 50 or fewer, 20 or fewer, 10 or fewer, 5 or fewer, or even one analyte.

The terms “analyte” and “target analyte” are used herein interchangeably and refer to a known or unknown component of a sample, which specifically binds to a capture agent if the analyte and the capture agent are members of a specific binding pair. Any convenient analytes may be targeted for detection according to the subject methods. In some cases, analytes are biomolecules, e.g., biopolymers, e.g., an oligomer or polymer such as an oligonucleotide, a peptide, a polypeptide, or an antibody. Additional analytes of interest include lipids, phospholipids, hormones, neurotransmitters, sugars or metabolites, whole cells, cellular components, viruses, macromolecular complexes (such as lipoproteins, ribosomes, nucleosomes), drugs, toxins, small molecules or environmental contaminants, and the like. In some cases, an analyte is referenced as a moiety in a mobile phase (in some cases fluid), to be detected by a capture agent and a reporter complex.

A “biopolymer” is a polymer of one or more types of repeating units, regardless of the source. Biopolymers may be found in biological systems and may include polypeptides, polynucleotides, sugars, carbohydrates, and analogs thereof.

As used herein, the term “polypeptide” refers to a polymeric form of amino acids of any length, including peptides that range from 2-50 amino acids in length and polypeptides that are greater than 50 amino acids in length. The terms “polypeptide” and “protein” are used interchangeably herein. The term “polypeptide” includes polymers of coded and non-coded amino acids, chemically or biochemically modified or derivatized amino acids, and polypeptides having modified peptide backbones in which the conventional backbone has been replaced with non-naturally occurring or synthetic backbones. A polypeptide may be of any convenient length, e.g., 2 or more amino acids, such as 4 or more amino acids, 10 or more amino acids, 20 or more amino acids, 50 or more amino acids, 100 or more amino acids, 300 or more amino acids, such as up to 500 or 1000 or more amino acids. “Peptides” may be 2 or more amino acids, such as 4 or more amino acids, 10 or more amino acids, 20 or more amino acids, such as up to 50 amino acids. In some embodiments, peptides are between 5 and 30 amino acids in length. The term “polypeptide” includes fusion proteins, including, but not limited to, fusion proteins with a heterologous amino acid sequence, fusions with heterologous and native leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like. In some cases, a protein may be composed of two or more peptides and/or polypeptides.

As used herein the term “isolated,” refers to a moiety of interest that is at least 60% free, at least 75% free, at least 90% free, at least 95% free, at least 98% free, and even at least 99% free from other components with which the moiety is associated with prior to purification.

The terms “nucleic acid,” “nucleic acid molecule”, “oligonucleotide” and “polynucleotide” are used interchangeably and refer to a polymeric form of nucleotides of any length, either deoxyribonucleotides or ribonucleotides, or compounds produced synthetically which can hybridize with naturally occurring nucleic acids in a sequence specific manner similar to that of two naturally occurring nucleic acids, e.g., can participate in Watson-Crick base pairing interactions. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, cDNA, recombinant polynucleotides, plasmids, vectors, isolated DNA of any sequence, control regions, isolated RNA of any sequence, nucleic acid probes, primers and any convenient synthetic nucleic acid sequence. The term “polynucleotide” is also meant to encompass nucleic acid analogs, and mixtures of analogs and naturally occurring nucleic acids. Any kind of nucleic acid, such as DNA and RNA, capable of sequence specific hybridization through formation of base pairs—or similar interactions between two moieties—may be utilized to implement the methods described herein, including artificial and unnatural nucleic acid analogs such as PNA, LNA, MNA, ANA, TNA, CeNA, GNA, XNA, HNA, INA, BNA and bicyclo-DNA. Sequence specific pairing of polynucleotides of interest that find use in the subject methods may involve natural Watson-Crick base pairing, Hoogsteen pairing, metal ion pairing, or other configurations or pairings between base moieties forming hydrogen bonds, metal ion interactions, or other types of moieties forming sequence specific pairing interactions such as unnatural base pairs (UBP) that may involve hydrogen bonds, hydrophobic interactions or other types of non-covalent bonds.

Specific pairing interactions of polynucleotides may involve natural, unnatural, artificial or modified bases. Analogs or moieties of interest include, but are not limited to, adenine, guanine, thymidine, cytosine, uridine, inosine, thiouridine, 5-bromouracil, methylated bases, 5-methylcytocine and 5-hydroxymethylcytocine, diaminopurine, diaminopyridine, isoguanine, isocytosine, 2′-deoxyinosine, 2-aminoadenine, xanthine, beta-d-glucopyranosyloxymethyluracil, d5SICS, dNaM, 2-amino-8-(2-thienyl)purine, pyridine-2-one, 7-(2-thienyl)imidazo[4,5-b]pyridine, pyrrole-2-carbaldehyde, 4-[3-(6-aminohexanamido)-1-propynyl]-2-nitropyrrole, 2,4-difluorotoluene, 4-methylbenzimidazole, isoquinoline, pyrrolo[2,3-b]pyridine, 2,6-bis(ethylthiomethyl)pyridine, pyridine-2,6-dicarboxamide, and mondentate pyridine.

Nucleic acid analogs of interest may include any convenient combination of backbones, bases (or analogs thereof), and pairing moieties that result in a molecule capable of sequence specific binding with a complementary nucleic acid analog of the same or different type which contains a complementary sequence in at least a portion of its sequence.

The term “sequence” may refer to a particular sequence of bases and/or may also refer to a polynucleotide having a particular sequence of bases. Thus a sequence may be information or may refer to a molecular entity, as indicated by the context of the usage.

The term “moiety” is used to refer to a portion of an entity or molecule, in some cases having a particular function, structure, or structural feature.

The terms “detectable moiety”, “detectable tag” and “measurable moiety” are used interchangeably herein to refer to a tag, moiety, and/or molecule which has properties that can be detected and/or measured, directly or indirectly.

The terms “antibody,” “immunoglobulin” and their plural referents include antibodies or immunoglobulins of any isotype, fragments of antibodies which retain specific binding to antigen, including, but not limited to, Fab, Fv, scFv, and Fd fragments, chimeric antibodies, humanized antibodies, single-chain antibodies, and fusion proteins including an antigen-binding portion of an antibody and a non-antibody protein. The antibodies may be bound to an entity that enables their detection, e.g., a radioisotope, an enzyme which generates a detectable product, a fluorescent protein, and the like. The antibodies may be further covalently or non-covalently conjugated to other moieties, such as members of specific binding pairs, e.g., biotin (member of biotin-avidin/streptavidin specific binding pair), and the like. The antibodies may also be bound to a solid support, including, but not limited to, polystyrene plates or beads, and the like. Also encompassed by the terms are Fab′, Fv, F(ab′)2, and or other antibody fragments that retain specific binding to antigen. Antibodies may exist in a variety of other forms including, for example, Fv, Fab, and (Fab′)2, as well as bi-functional (i.e. bi-specific) hybrid antibodies (e.g., Lanzavecchia et al., Eur. J. Immunol. 17, 105 (1987)) and in single chains (e.g., Huston et al., Proc. Natl. Acad. Sci. USA, 85, 5879-5883 (1988); Bird et al., Science, 242, 423-426 (1988); see Hood et al., Immunology, Benjamin, N.Y., 2nd ed. (1984), and Hunkapiller and Hood, Nature, 323, 15-16 (1986)).

The terms “capable of hybridizing,” “hybridizing”, and “hybridization” as used herein refers to binding between complementary or partially complementary molecules, for example as between the sense and anti-sense strands of double-stranded DNA. Such binding is commonly non-covalent binding, and is specific enough such that binding may be used to differentiate between highly complementary molecules and others less complementary. Examples of highly complementary molecules include complementary oligonucleotides, DNA, RNA, and the like, which include a region of nucleotides arranged in the nucleotide sequence that is exactly complementary to a second nucleic acid sequence; examples of less complementary oligonucleotides include ones with nucleotide sequences including one or more nucleotides not in the sequence exactly complementary to a second oligonucleotide.

The term “complementary” references a property of specific binding between pairs of specific binding moieties. Specific binding moieties are complementary if they specifically bind to each other. A pair of specific binding moieties that are each polynucleotides (including naturally occurring nucleic acids and nucleic acid analogs) may be complementary based on their sequence complementarity. In some cases, polynucleotides are complementary if they bind to each other in a hybridization assay under stringent conditions. Portions of polynucleotides are complementary to each other if they follow conventional base-pairing rules, e.g. A pairs with T (or U) and G pairs with C, or if they follow any convenient sequence specific pairing interactions such as unnatural base pairs (UBP) that may involve hydrogen bonds, hydrophobic interactions or other types of non-covalent bonds. “Complementary” includes embodiments in which there is an absolute sequence complementarity, and also embodiments in which there is a substantial sequence complementarity. Additional examples of specific binding pairs which may be considered complementary include antibody-antigen binding pairs, receptor-ligand binding pairs, nucleic acid aptamer-protein binding pairs and the like.

“Absolute sequence complementarity” means that there is 100% sequence complementarity between a first polynucleotide and a second polynucleotide, i.e. there are no insertions, deletions, or substitutions in either of the first and second polynucleotides with respect to the other polynucleotide (over the complementary region). Put another way, every base (or analog thereof) of the complementary region is paired with its complementary base (or analog thereof) by base-pairing or other specific pairing as described herein.

“Substantial sequence complementarity” permits one or more relatively small (in some cases, less than 10 bases, e.g. less than 5 bases, typically less than 3 bases, more typically a single base) insertions, deletions, or substitutions in the first and or second polynucleotide (over the complementary region) relative to the other polynucleotide. The complementary region is the region that is complementary between a first polynucleotide and a second polynucleotide (e.g. a distinct sequence of a nucleic acid target molecule and a nucleic acid capture agent). Complementary sequences are in some cases embedded within larger polynucleotides, thus two relatively long polynucleotides may be complementary over only a portion of their total length. The complementary region may be of any convenient length, and is in some cases at least 5 bases long, such as at least 7 bases long, at least 12 bases long, at least 15 bases long, at least 20 bases long, at least 25 bases long, at least 30 bases long, at least 40 bases long, at least 50 bases long, at least 60 bases long, at least 70 bases long, at least 80 bases long, at least 90 bases long, at least 100 bases long, at least 200 bases long, at least 300 bases long, at least 400 bases long, at least 500 bases long, at least 600 bases long, at least 700 bases long, at least 800 bases long, at least 1000 bases long, at least 2000 bases long, at least 3000 bases long, at least 4000 bases long, at least 5000 bases long, or even longer.

The terms “hybridizing specifically to,” “specific hybridization,” “selectively hybridize to,” and the like are used herein to refer to the binding, duplexing, or hybridizing of a nucleic acid molecule preferentially to a particular nucleotide sequence under “stringent conditions.”

The term “stringent conditions” refers to conditions under which a first molecule, e.g., a first nucleic acid, will bind preferentially to a second molecule, e.g., a second nucleic acid, and to a lesser extent to, or not at all to, e.g., other sequences. Put another way, the term “stringent hybridization conditions” as used herein refers to conditions that are compatible to produce complexes (e.g., duplexes) between complementary binding members, e.g., between a sequence of a nucleic acid capture agent and a complementary sequence of a target nucleic acid. In some instances, the first and second complementary binding members include molecules selected from a protein, such an antibody, which specifically binds to a complementary antigen and not to other molecules under stringent conditions. Stringent conditions for specific binding involving biomolecules such as proteins may include high salt concentrations and high temperatures.

“Stringent hybridization conditions” and “stringent hybridization wash conditions” in the context of nucleic acid hybridization are sequence dependent, and are different under different environmental parameters. Stringent hybridization conditions can include, e.g., hybridization in a buffer including 50% formamide, 5× saline sodium citrate (SSC), and 1% sodium dodecyl sulfate (SDS) at 42° C., or hybridization in a buffer including 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Yet additional stringent hybridization conditions include hybridization at 60° C. or higher and 3×SSC (450 mM NaCl/45 mM sodium citrate) or incubation at 42° C. in a solution containing 30% formamide, 1M NaCl, 0.5% sodium sarcosine, 50 mM 2-(N-morpholino)ethanesulfonic acid, pH 6.5. Those of ordinary skill will readily recognize that alternative but comparable hybridization and wash conditions can be utilized to provide conditions of similar stringency.

In certain embodiments, the stringency of the wash conditions may affect the degree to which nucleic acid molecules specifically hybridize. Suitable wash conditions may include, e.g.: a salt concentration of about 0.02 M at pH 7 and a temperature of at least about 50° C. or about 55° C. to about 60° C.; or, a salt concentration of about 0.15 M NaCl at 72° C. for about 15 min; or, a salt concentration of about 0.2×SSC at a temperature of at least about 50° C. or about 55° C. to about 60° C. for about 1 to about 20 min; or, multiple washes with a solution with a salt concentration of about 0.1×SSC containing 0.1% SDS at 20 to 50° C. for 1 to 15 min; or, equivalent conditions. Stringent conditions for washing can also be, e.g., 0.2×SSC/0.1% SDS at 42° C. In instances wherein the nucleic acid molecules are oligodeoxynucleotides (i.e. oligonucleotides made up of deoxyribonucleotide subunits), stringent conditions can include washing in 6×SSC/0.05% sodium pyrophosphate at 37° C. (for 14-base oligos), 48° C. (for 17-base oligos), 55° C. (for 20-base oligos), and 60° C. (for 23-base oligos). See Sambrook, et al., Molecular Cloning: A Laboratory Manual, Second Edition, (1989) Cold Spring Harbor, N.Y.), for detailed descriptions of equivalent hybridization and wash conditions and for reagents and buffers, e.g., SSC buffers and equivalent reagents and conditions.

Stringent hybridization conditions may also include a “prehybridization” of aqueous phase nucleic acids with complexity-reducing nucleic acids to suppress repetitive sequences. For example, certain stringent hybridization conditions include, prior to any hybridization to surface-bound polynucleotides, hybridization with random sequence synthetic oligonucleotides (e.g. 25-mers), or the like. Other stringent hybridization conditions are known in the art and may also be employed, as appropriate.

The terms “bind” and “bound” as used herein refer to a binding interaction between two or more entities. Where two entities, e.g., molecules, are bound to each other, they may be directly bound, i.e., bound directly to one another, or they may be indirectly bound, i.e., bound through the use of an intermediate linking moiety or entity. In either case the binding may be covalent; e.g., through covalent bonds; or non-covalent, e.g., through ionic bonds, hydrogen bonds, electrostatic interactions, hydrophobic interactions, Van der Waals forces, or a combination thereof.

The terms “specific binding,” “specifically bind,” and the like, refer to the ability of a first binding molecule or moiety (e.g., a target-specific binding moiety such as a capture agent or a first specific binding moiety) to preferentially bind directly to a second binding molecule or moiety (e.g., a target molecule or a second specific binding moiety) relative to other molecules or moieties in a reaction mixture. In certain embodiments, the affinity between a first binding molecule or moiety and a second binding molecule or moiety when they are specifically bound to each other is characterized by a K_{D} (dissociation constant) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. In some cases, the affinity between a capture agent and analyte when they are specifically bound in a capture agent/analyte complex is at least 10⁻⁸ M, at least 10⁻⁹ M, or at least 10⁻¹⁰ M. In some instances, a specific binding interaction will discriminate between desirable and undesirable analytes in a sample with a specificity of 10-fold or more for a desirable analyte over an undesirable analytes, such as 100-fold or more, or 1000-fold or more.

As used herein, a “member of a specific binding pair” is a member of a pair of molecules or entities that takes part in a specific binding interaction. Where a first member of the specific binding pair is identified, the identity of the second member of the specific binding pair may be readily identifiable. It should be noted that when either member of the binding pair is referred to as the first member, the remaining member is understood to be the second member and vice versa. Examples of specific binding pair interactions include immune interactions such as antigen/antibody and hapten/antibody as well as non-immune interactions such as complementary nucleic acid binding, complementary protein-protein interactions, a sugar and a lectin specific therefore, an enzyme and an inhibitor therefore, an apoenzyme and cofactor, a hormone and a receptor therefore, biotin/avidin and biotin/streptavidin.

As used herein, the term “biotin moiety” refers to an affinity agent that includes biotin or a biotin analogue such as desthiobiotin, oxybiotin, 2′-iminobiotin, diaminobiotin, biotin sulfoxide, biocytin, etc. Biotin moieties bind to streptavidin with an affinity of at least 10⁻⁸M. A biotin affinity agent may also include a linker, e.g., -LC-biotin, -LC-LC-Biotin, -SLC-Biotin or -PEGₙ-Biotin where n is 3-12.

As used herein, the terms “chemoselective functional group” and “chemoselective group” are used interchangeably and refer to chemoselective reactive groups that selectively react with one another to form a covalent bond. Chemoselective functional groups of interest include, but are not limited to, thiols and maleimide or iodoacetamide, as well as groups that can react with one another via Click chemistry, e.g., azide and alkyne groups (e.g., cyclooctyne groups).

As used herein, the term “magnetic” in “magnetic particle” refers to all subtypes of magnetic particles, where examples of subtypes of magnetic particles include, but are not limited to, ferromagnetic particles, superparamagnetic particles and paramagnetic particles. “Ferromagnetic” materials are strongly susceptible to magnetic fields and are capable of retaining magnetic properties when the field is removed. “Paramagnetic” materials have only a weak magnetic susceptibility and when the field is removed quickly lose their weak magnetism. “Superparamagnetic” materials are highly magnetically susceptible, i.e. they become strongly magnetic when placed in a magnetic field, but, like paramagnetic materials, rapidly lose their magnetism.

The methods described herein include multiple steps. Each step may be performed after a predetermined amount of time has elapsed between steps, as desired. As such, the time between performing each step may be 1 second or more, 10 seconds or more, 30 seconds or more, 60 seconds or more, 5 minutes or more, 10 minutes or more, 60 minutes or more and including 5 hours or more. In certain embodiments, each subsequent step is performed immediately after completion of the previous step. In other embodiments, a step may be performed after an incubation or waiting time after completion of the previous step, e.g., a few minutes to an overnight waiting time.

It will be appreciated by those of skill in the art that many of the above-provided definitions overlap in scope and are not meant to be mutually exclusive. Accordingly, any particular chemical group may fall within more than one of the above-provided definitions.

Before the present invention is further described, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a capture agent” includes a plurality of such capture agents and reference to “the reporter complex” includes reference to one or more reporter complexes and equivalents thereof known to those skilled in the art, and so forth. It is further noted that the claims may be drafted to exclude any element, e.g., any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Further, the dates of publication provided may be different from the actual publication dates which may need to be independently confirmed.

The following Detailed Description is put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius (° C.), and pressure is at or near atmospheric. Standard abbreviations may be used, e.g., bp, base pair(s); kb, kilobase(s); microliter(s); pl, picoliter(s); μm, micrometer/micron; s or sec, second(s); min, minute(s); h or hr, hour(s); aa, amino acid(s); nt, nucleotide(s); and the like.

### DETAILED DESCRIPTION

As summarized above, the present disclosure provides methods of detecting a target analyte in a sample, and compositions for practicing the same. The target analyte may be captured from the sample via the formation of a sandwich complex with first and second capture agents that specifically bind the target analyte. The first capture agent may be attached to a substrate to facilitate separation and the second capture agent may be part of a reporter complex to facilitate detection. The methods of detection may involve the release of a detectable tag from the reporter complex, where the amount of detectable tag released is proportional to the amount of the target analyte bound to the reporter complex. The detectable tag may be released from the reporter complex using a displacement binding member that disrupts a binding interaction between a pair of specific binding members in the reporter complex. For example, in some cases, the displacement binding member displaces or releases the detectable tag from the reporter complex by means of polynucleotide strand displacement of a specific binding member. In some cases, release of the detectable tag is achieved via cleavage. The method may further include releasing the detectable tag in a concentrated form thus providing an improved limit of detection (LOD) relative to direct detection of the target analyte. The subject methods and compositions achieve release (e.g., displacement or cleavage) of a detectable tag from a reporter complex under mild conditions and using stable components, where the target analyte is not the mediator of release. Any type of target analytes such as nucleic acids, proteins, hormones, sugars and many others can be detected and analyzed using the subject methods. The reporter complex and displacement binding agent may be utilized universally with and adapted for different target analytes.

Each of these components that find use in the subject methods and compositions are now described in more detail, followed by further details of the methods of using the same.

### Target Analytes

Any convenient samples (e.g., as defined herein) may be analyzed according to the subject methods. The sample may include, or be suspected of including, one or more target analytes of interest. The compositions and methods of the present disclosure may be utilized in connection with the qualitative and/or quantitative detection of any of a wide variety of target molecules or other analytes of interest. Target analytes of interest include, but are not limited to, a nucleic acid, such as an RNA, DNA, PNA, CNA, HNA, LNA or ANA molecule, a protein, such as a fusion protein, a modified protein, such as a phosphorylated, glycosylated, ubiquitinated, SUMOylated, or acetylated protein, an antibody or a fragment thereof (including single-chain antibodies, Fabs, and the like), a peptide, an aggregated biomolecule, viruses, whole cells, cellular components, organic and inorganic small molecules, lipids, sugars, hormones, a vitamin and a drug molecule. As used herein, the term “a target protein” refers to all members of the target protein family, and fragments thereof. The target protein may be any protein of interest, such as a therapeutic or diagnostic target, including but not limited to: hormones, growth factors, receptors, enzymes, cytokines, osteoinductive factors, colony stimulating factors and immunoglobulins. The term “target protein” is intended to include recombinant and synthetic molecules, which can be prepared using any convenient recombinant expression methods or using any convenient synthetic methods, or purchased commercially. In some embodiments, the target analyte is a nucleic acid, a protein, a hormone, a lipid or a sugar. Protein targets of interest include, for example, cell surface receptors, signal transduction factors, and hormones. Nucleic acid targets of interest include, for example, DNA and RNA targets. Cellular targets of interest include, for example, mammalian cells (particularly human cells, e.g., human cancer cells) stem cells, and bacterial cells.

In some embodiments, the target analyte is a biomarker of disease. A “biomarker,” as used herein, is any molecule or compound that is found in a sample of interest and that is known to be diagnostic of or associated with the presence of or a predisposition to a disease or condition of interest in the subject from which the sample is derived. Biomarkers include, but are not limited to, polypeptides or a complex thereof (e.g., antigen, antibody), nucleic acids (e.g., DNA, miRNA, mRNA), drug metabolites, lipids, carbohydrates, hormones, vitamins, etc., that are known to be associated with a disease or condition of interest.

### Capture Agents

Aspects of the subject methods include contacting the sample with: a first capture agent that specifically binds a target analyte; and a reporter complex that includes a second capture agent that also specifically binds the target analyte, to form a sandwich complex. The first and second capture agents may be moieties that are each capable of specifically binding to a target analyte of interest when both brought into contact with the analyte under suitable reaction conditions. The binding interaction is, in some cases, mediated by an affinity region of the capture agent and a complementary affinity region of the target analyte. Any convenient capture agents may be selected as first and second capture agents and utilized to specifically bind a target analyte into a sandwich complex. In some cases, the second capture agent is itself part of a larger complex, such as a reporter complex, that includes additional components which do not specifically interact with the target. In certain embodiments of the method, the sample is contacted with: a first capture agent that specifically binds a target analyte; and a second capture agent that also specifically binds the target analyte, to form a sandwich complex, where the second capture agent is capable of forming a reporter complex (e.g., as described herein). The reporter complex may be formed before or after formation of the sandwich complex. In some cases, one or more components (e.g., as described herein) of the reporter complex are contacted with the sandwich complex and specifically bind to the second capture agent. Capture agents of interest include, but are not limited to, proteins such as antibodies, scaffolded protein ligands or proteins involved in known biomolecule interactions (e.g., polynucleotide binding proteins or protein-protein interactions), polynucleotides such as aptamers or polynucleotides with complementary sequences, peptides, enzyme substrates, antigens, haptens, small molecules, inhibitors, or an analog thereof. In some embodiments, the first capture agent and the second capture agent are independently selected from a nucleic acid (e.g., an aptamer or complementary polynucleotide sequence), a protein (e.g., an antibody), a peptide, and a small molecule (e.g., a hapten).

Target-specific capture agents may have a variety of structures provided that they are capable of specifically binding to a target analyte of interest under suitable reaction conditions. For example, where the target molecule is a nucleic acid, a suitable target-specific capture agent may be a nucleic acid molecule having a region of sequence complementarity to a region of the target nucleic acid molecule, e.g., a region of substantial or absolute sequence complementarity. Where the target is a protein or fragment thereof a suitable target-specific capture agent may be an antibody capable of specifically binding to the target molecule. Additional binding members capable of specific interactions are known in the art and accordingly a suitable target-specific capture agent may be readily identified and prepared for a specific target molecule or analyte of interest using standard techniques.

The first and second capture agents may be selected to form a desired sandwich complex with the target analyte. The term sandwich complex is meant to include any complex that includes at least the three desired components of a target analyte, and first and second capture agents. The sandwich complex may be formed via any convenient sequence of binding steps. In some instances, specific binding of a second capture agent is dependent on prior formation of a complex between the target analyte and a first capture agent, or vice versa. In certain cases, the first and second capture agents may independently and specifically bind to distinct affinity regions of the target analyte, which may be achieved simultaneously or via sequential binding steps. Any convenient sandwich complexes may be adapted for use in the subject methods. Sandwich complexes of interest include, but are not limited to, antibody sandwich complexes including a target protein of interest, aptamer sandwich complexes including a target protein of interest, antibody-aptamer sandwich complexes including a target protein of interest, protein sandwich complexes including a target polynucleotide of interest, sandwich complexes including a small molecule ligand or inhibitor for a target protein and sandwich complexes including a target polynucleotide and at least one polynucleotide affinity agent.

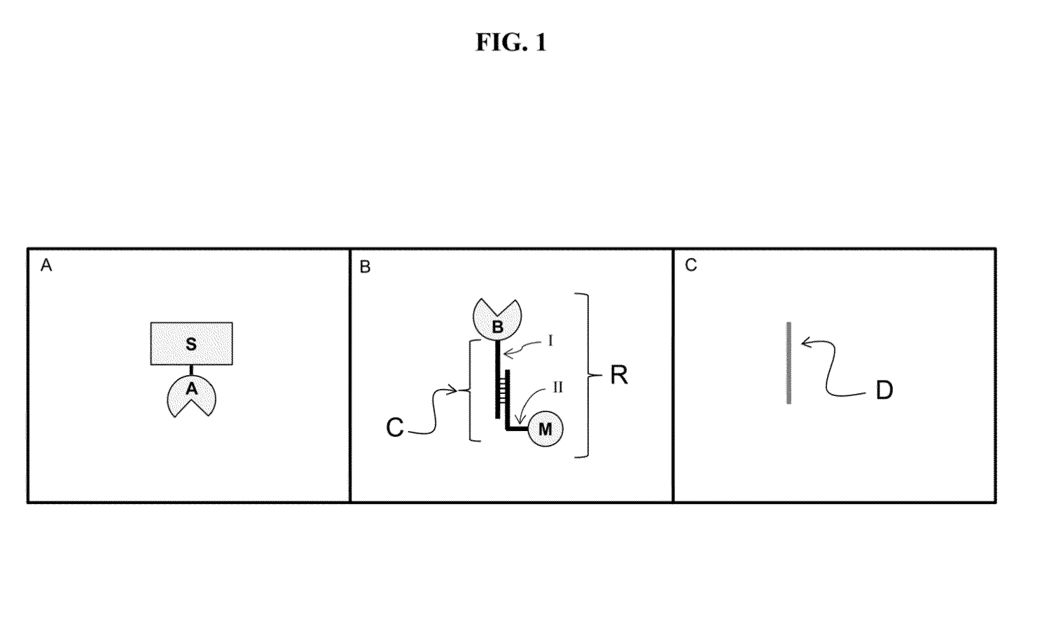

The first capture agent may further provide for separation of the target analyte from the sample. In some cases, the first capture agent is linked to a convenient moiety that facilitates separation of the target analyte from the sample, where the linkage may be covalent or non-covalent (see, e.g., FIG. 1). In some cases, the first capture agent is linked to a support (e.g., directly or indirectly). In certain cases, the first capture agent is linked to a polymer (e.g., a polymer capable of acting as a liquid or solid support). In certain instances, the first capture agent is linked to a chemoselective group (e.g., a group that provides for subsequent immobilization of the capture agent). In some cases, the term “support bound” refers to a covalent linkage to the surface of a solid support. Use of a support bound capture agent provides for immobilization and/or separation of any target analyte to which the capture agent binds. A variety of methods may be utilized to separate a target analyte from a sample via immobilization on a support. Any convenient supports may be utilized in the subject methods to immobilize the sandwich complex. Supports of interest include, but are not limited to: solid substrates, where the substrate can have a variety of configurations, e.g., a sheet, bead, or other structure, such as a plate with wells; beads, polymers, particles (e.g., cells or magnetic beads), a fibrous mesh, hydrogels, porous matrix, a pin, a microarray surface, a chromatography support, and the like. In some instances, the support is selected from the group consisting of a particle, a planar solid substrate, a fibrous mesh, a hydrogel, a porous matrix, a pin, a microarray surface and a chromatography support. In some instances, the support is a biological particle such as a viral particle or bacteriophage. Such supports can be separated from a sample or other aqueous mixture by any convenient method, such as by precipitation, sedimentation, affinity capture, filtration, etc. The support may be incorporated into a system that provides for target isolation assisted by any convenient methods, such as a manually-operated syringe, a centrifuge or an automated liquid handling system. In certain instances, the support includes a magnetic particle. In some cases, the support is composed of colloidal magnetic particles. The term “particle” as used herein refers to a solid phase such as colloidal particles, microspheres, nanoparticles, or beads. In some cases, the particle may have a size in diameter ranging from 10 nm to 1000 μm, such as from 100 nm to 900 μm, 200 nm to 800 μm, 300 nm to 700 μm, 400 nm to 600 μm, 500 nm to 500 μm, 600 nm to 400 μm, 700 nm to 300 μm, 800 nm to 200 μm, 900 nm to 100, or 1000 nm to 10 μm. In some embodiments, the particle may have a size in diameter ranging from 10 nm to 1 μm, from 1 μm to 100 μm, from 100 μm to 500 μm, or from 500 μm to 1000 μm. In some embodiments, the particle may have a size in diameter ranging from 10 nm to 1400 nm, such as from 100 to 1400 nm, 200 to 1300 nm, 300 nm to 1200 nm, 400 nm to 1100 nm, 500 nm to 1000 nm, 600 nm to 900 nm, or 700 nm to 800 nm. In some embodiments, the particle may have a size in diameter ranging from 10 nm to 1000 nm, 10 nm to 500 nm, 10 nm to 400 nm, 10 nm to 300 nm, 10 nm to 200 nm, or 10 nm to 100 nm. In some embodiments, a suitable particle may have a diameter of from about 100 nm to about 200 nm, about 200 nm to about 300 nm, about 300 nm to about 400 nm, about 400 nm to about 500 nm, about 500 nm to about 600 nm, about 600 nm to about 700 nm, about 700 nm to about 800 nm, about 800 nm to about 900 nm, about 900 nm to about 1000 nm, about 1000 nm to about 1100 nm, about 1100 nm to about 1200 nm, about 1200 nm to about 1300 nm, or about 1300 nm to about 1400 nm. In certain instances, a first capture agent (e.g., as described herein) is linked to the magnetic particle. FIG. 1 illustrates a support bound first capture agent that specifically binds a target analyte. In some embodiments, the first capture agent is linked to a support such as a bead, a particle (e.g., a magnetic particle), a gel, a membrane, a fiber, a biosensor chip surface, a vessel (e.g., a tube surface), a cell, (e.g., a bacterium), a polymer or other suitable support.

### Specific Binding Members

The reporter complex may further include a first specific binding member linked to a capture agent that specifically binds the target analyte (e.g., as described herein). The first specific binding member is complementary to a second specific binding member to which it is specifically bound in the reporter complex. The first and second specific binding members may be non-covalently bound to each other in the reporter complex. Any convenient pairs of specific binding members may be utilized in the subject reporter complexes. Specific binding members of interest include, but are not limited to, proteins such as antibodies, scaffolded protein ligands or proteins involved in known biomolecule interactions (e.g., polynucleotide binding proteins or protein-protein interactions), polynucleotides (e.g., DNAs, RNAs, PNAs and mixtures thereof) such as aptamers or polynucleotides with complementary sequences, peptides, enzyme substrates, antigens, haptens, small molecules, inhibitors, or an analog thereof. Pairs of specific binding members of interest include, pairs of complementary (full or partially complementary) polynucleotide sequences (see e.g., FIG. 1), antigen-antibody pairs, hapten-antibody pairs, enzyme-inhibitor pairs, protein-protein interaction pairs, biotin moiety and avidin moiety pairs, aptamer-protein pairs, etc.

### Displacement Binding Members

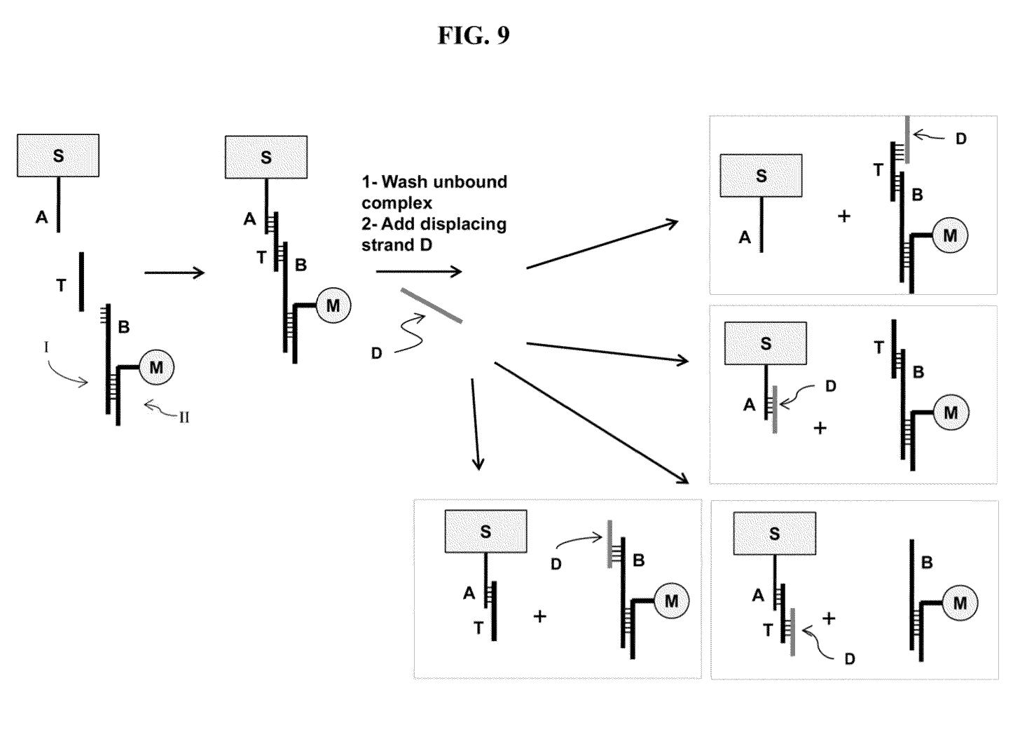

In some cases, the second specific binding member may be released from the reporter complex specifically by disrupting the binding of the first and second specific binding members. In some cases, “releasing” may be described as “displacing”. Displacement of a specific binding member from the complex may be achieved by contacting the complex with a competitive binder and/or a binder which physically displaces one of the first and second specific binding members, e.g., a displacement binding member. Any convenient binding interactions of the complex, including the sandwich complex and the reporter complex may be targeted for disruption by a displacement binding member (see e.g., FIG. 9). It should be understood that this applies to any suitable embodiment described herein. In certain instances, the displacement binding member is complementary to the first or second specific binding member. In certain instances, the displacement binding member is complementary to the first or second capture agent. In certain instances, the displacement binding member is complementary to the first capture agent. In certain instances, the displacement binding member is complementary to the second capture agent. In certain instances, the displacement binding member is complementary to the target, e.g., one of the sites of the target that specifically binds to the first or second capture agent. In certain instances, the displacement binding member is complementary to one of a pair of specific binding members that links (e.g., indirectly) the second specific binding member to the detectable tag (see e.g., FIG. 9). By complementary is meant that the displacement binding member is capable of specifically binding the first or second specific binding member, and as such, the displacement binding member may compete for binding with the other specific binding member of the pair and/or physically displace the other specific binding member of the pair. In some cases, the displacement binding member is a complementary polynucleotide (e.g., a polynucleotide having sequence complementarity to a specific binding member that is a polynucleotide). In some embodiments, where the displacement binding member is a polynucleotide, the region of complementarity between the displacement binding member and a first member of the sandwich complex or reporter complex to which it binds is longer than the region of complementarity between the first member of the sandwich complex or reporter complex and a second member of the sandwich complex or reporter complex which is displaced by the displacement binding member. In certain cases, the displacement binding member is a complementary polypeptide. In certain instances, the displacement binding member is a complementary small molecule. The first and second specific binding members and the displacement binding member may be selected to provide for release of the second specific binding member from the receptor complex. In some embodiments, the first specific binding member, the second specific binding member and the displacement binding member are independently selected from a nucleic acid, a protein, a peptide, a small molecule, or an analog thereof (e.g., an antibody, a hapten, an aptamer or a polynucleotide). In certain embodiments, the first specific binding member, the second specific binding member and the displacement binding member are each independently a nucleic acid (e.g., a DNA, a RNA or a PNA or a nucleic acid analog). See, for example, FIG. 1 where components that find use in one embodiment of the subject method are depicted. While the Figures depict the first and second specific binding members and displacement binding members as nucleic acids, such depiction is for the purposes of illustration only. It should be noted that any of the above combinations of specific binding members and displacement binding members may be utilized in such embodiments provided that they provide for the binding and displacement functionality described herein.

Any convenient displacement binding members capable of disrupting the complex may be utilized in the subject methods. Displacement binding members of interest include, but are not limited to, proteins such as antibodies, scaffolded protein ligands or proteins involved in biomolecule interactions (e.g., polynucleotide binding proteins or protein-protein interactions), polynucleotides (e.g., DNAs, RNAs, PNAs, modified nucleic acid analogs, and mixtures thereof) such as aptamers or polynucleotides with complementary sequences, peptides, enzyme substrates, antigens, haptens, small molecules, inhibitors, and the like.

In some embodiments, the displacement binding member is complementary to the first specific binding member of a reporter complex. In certain embodiments, the displacement binding member is capable of hybridizing to the first specific binding member, thereby releasing the second specific binding member from the reporter complex. In some embodiments, the displacement binding member is complementary to the second specific binding member of a reporter complex. In certain embodiments, the displacement binding member is capable of hybridizing to the second specific binding member, thereby releasing the second specific binding member from the reporter complex.

### Detectable Tags

Aspects of the method further include detecting a detectable tag that may be released with, or that is an integral part of, a specific binding member that is released from the reporter complex. The detectable tag is any convenient moiety that may be detected directly or indirectly using any convenient means. In certain embodiments, the specific binding member is itself detectable. In some embodiments, the second specific binding member is linked (e.g., covalently or non-covalently) to a detectable tag. As such, the second specific binding member may include a detectable tag (e.g., may itself be detectable or may be linked thereto). The detectable tag may be detected directly, e.g., by fluorescence, luminescence or radioactivity or indirectly, e.g., by a subsequent enzyme-catalyzed reaction or by subsequent chemoselective labelling with a detectable tag. In some instances, detecting the detectable tag comprises: identifying a nucleic acid or polypeptide, detecting a fluorescent, luminescent or radioactive signal, detecting the product of an enzyme-catalyzed reaction or chemoselectively attaching a fluorophore to a chemoselective tag. In some embodiments, the specific binding member is a polynucleotide that is itself a detectable tag that may be detected using any convenient method, such as a nucleic acid sequencing method.

Any convenient detectable tags may be utilized in the subject methods. Detectable tags of interest include, but are not limited to, an enzyme, a nucleic acid, a polypeptide, a particle, an affinity tag (e.g., an antigen, a hapten or a member of a specific binding pair such as a biotin moiety), a fluorophore, a chromophore, a luminescent tag, a radioactive tag or a chemoselective tag.

There are a variety of detectable tags known in the art which can be utilized in connection with the disclosed methods and compositions. These include, for example, fluorophores, chromophores, luminescent labels, metal complexes, radioisotopes, polynucleotides, specific binding moieties such as a biotin moiety, an antigen or a peptide, enzymes (e.g., peroxidases (e.g., horseradish peroxidase), glycosidases and phosphatases (e.g., alkaline phosphatase)), fluorescent particles, chemiluminescent particles and magnetic particles.

Fluorophores of interest that find use as detectable tags in the subject methods and compositions, include but are not limited to, fluorescein, 6-FAM, rhodamine, Texas Red, tetramethylrhodamine, carboxyrhodamine, carboxyrhodamine 6G, carboxyrhodol, carboxyrhodamine 110, Cascade Blue, Cascade Yellow, coumarin, Cy2, Cy3, Cy3.5, Cy5, Cy5.5, Cy7, Cy-Chrome, phycoerythrin, PerCP (peridinin chlorophyll-a Protein), PerCP-Cy5.5, JOE (6-carboxy-4′,5′-dichloro-2′,7′-dimethoxyfluorescein), NED, ROX (5-(and-6)-carboxy-X-rhodamine), HEX, Lucifer Yellow, Marina Blue, Oregon Green 488, Oregon Green 500, Oregon Green 514, Alexa Fluor 350, Alexa Fluor 430, Alexa Fluor 488, Alexa Fluor 532, Alexa Fluor 546, Alexa Fluor 568, Alexa Fluor 594, Alexa Fluor 633, Alexa Fluor 647, Alexa Fluor 660, Alexa Fluor 680, 7-amino-4-methylcoumarin-3-acetic acid, BODIPY FL, BODIPY FL-Br.sub.2, BODIPY 530/550, BODIPY 558/568, BODIPY 564/570, BODIPY 576/589, BODIPY 581/591, BODIPY 630/650, BODIPY 650/665, BODIPY R6G, BODIPY TMR, BODIPY TR, conjugates thereof, and combinations thereof. Exemplary lanthanide chelates include europium chelates, terbium chelates and samarium chelates.

Chemoselective functional groups or tags may be utilized in an indirect method of detection. In some cases, a detectable tag may include a chemoselective functional groups that is capable of reaction with a second compatible functional group, which reaction may provide for detection. Chemoselective functional groups that find use in the indirect detection of a detectable tag include, but are not limited to chemoselective functional groups selected from the group consisting of an alkyne, a cyclooctyne, an azide, a phosphine, a maleimide, a thiol, an alkoxyamine and an aldehyde.

In some embodiments of the methods and compositions disclosed herein, a detectable tag is utilized, wherein the detectable tag is a magnetic particle, e.g., a magnetic nano-particle or micro-particle. Magnetic particles include, for example, magnetic beads or other small objects made from a magnetic material such as a ferromagnetic material, a ferrimagnetic material, a paramagnetic material, or a superparamagnetic material. In some embodiments, the magnetic particles include iron oxide (Fe₂O₃ and/or Fe₃O₄) with diameters ranging from about 10 nm to about 100 μm. Magnetic nanoparticles are available, for example, from Miltenyi Biotec Corporation of Bergisch Gladbach, Germany. These are relatively small particles made from coated single-domain iron oxide particles, typically in the range of about 10 to about 100 nm in diameter. Magnetic particles can also be made from magnetic nanoparticles embedded in a polymer matrix such as polystyrene. Such particles may have diameters of about 1 to about 5 μm. Particles of this type are available from Invitrogen Corporation, Carlsbad, Calif. Additional examples of magnetic particles include those described by Baselt et al., Biosens. Bioelectron., 13, 731-739 (1998); Edelstein et al., Biosens. Bioelectron., 14, 805-813 (2000); Miller et al., J. of Mag. Magn. Mater., 225, 138-144 (2001); Graham et al., J. Appl. Phys., 91, 7786-7788 (2002); Ferreira et al. J. Appl. Phys., 93, 7281-7286 (2003); and U.S. Patent Application Publication No. 2005/0100930 (published May 12, 2005). In some embodiments, a detectable tag, e.g., a magnetic or non-magnetic particle, for use in connection with the present disclosure may have a diameter of from about 100 nm to about 50 μm, e.g., from about 200 nm to about 40 μm, from about 300 nm to about 30 μm, from about 400 nm to about 20 μm, or from about 500 nm to about 10 μm. In some embodiments, a detectable tag, e.g., a magnetic or non-magnetic particle, for use in connection with the present disclosure may have a diameter of from about 200 nm to about 20 μm, e.g., from about 300 nm to about 20 μm, from about 400 nm to about 20 μm, from about 500 nm to about 20 μm, from about 600 nm to about 20 μm, from about 700 nm to about 20 μm, from about 800 nm to about 20 μm, from about 900 nm to about 20 μm, from about 1 μm to about 20 μm, from about 2 μm to about 20 μm, from about 3 μm to about 20 μm, from about 4 μm to about 20 μm, from about 5 μm to about 20 μm, from about 6 μm to about 20 μm, from about 7 μm to about 20 μm, from about 8 μm to about 20 μm, from about 9 μm to about 20 μm, from about 10 μm to about 20 μm, from about 11 μm to about 20 μm, from about 12 μm to about 20 μm, from about 13 μm to about 20 μm, from about 14 μm to about 20 μm, from about 15 μm to about 20 μm, from about 16 μm to about 20 μm, from about 17 μm to about 20 μm, from about 18 μm to about 20 μm, or from about 19 μm to about 20 μm. In some cases, the particles have a diameter of from about 5 μm to about 50 μm, such as from about 10 μm to about 30 μm, from about 15 μm to about 25 μm, or about 20 μm.

A suitable detectable tag may be one which is detectable using a microscopy system which may or may not utilize specifically detectable characteristics of the detectable tag such as fluorescence or magnetic field. For example, in some embodiments, a suitable detectable tag may be detectable by an optical microscopy system based solely on its size and/or shape. This may be the case, for example, where a detectable particle is used which has a diameter of 0.1 μm or more, e.g., 0.5 μm or more, such as 5 μm or more, 10 μm or more, 20 μm or more, 50 μm or more, 100 μm or more, 100 μm or more, 200 μm or more, 300 μm or more, 400 μm or more, or 500 μm or more.

In some embodiments, a detectable tag particle suitable for use in connection with the disclosed methods and compositions includes covalent bond-forming reactive groups, e.g., chemoselective groups.

The detectable tag described herein may include members of a specific binding pair as defined previously herein. In some instances, the detectable tag is functionalized with molecules having binding properties that provide for a binding complex, e.g., a binding complex which serves to immobilize the detectable tag on a substrate surface, or a binding complex which serves to provide for a signal amplification system.

Once the detectable tag is released from the reporter complex or a portion thereof, one or more detection systems may be utilized to detect and or visualize the amount and/or location of the detectable tag which is indicative of the amount and/or location of the target molecule. The particular method used to detect the detectable tag will depend on the type of detectable tag utilized. For example, where the detectable tag is fluorescent, one or more fluorescence-based detection systems may be utilized. Where the detectable tag is a magnetic particle, one or more magnetic sensors may be utilized. Where the detectable tag finds use in an indirect detection method (e.g., an enzyme-catalyzed detection or a chemoselective tag), one or more additional steps detection steps and/or reagents may be utilized.

Detection devices and detection methods that may be used in connection with the disclosed methods may include well-plate based assays, ELISA, immunoassays, colorimetric assays, phosphogenic assays, luminogenic assays, fluorimetric assays, radionuclide-based assays, NMR, MS, any kind of spectrometry, CE, CZE, PAGE and other kinds of gel electrophoresis, biosensor-based devices, fluidic devices such as lab-on-chip, lateral flow devices, passive flow devices, chromatography, and any other suitable detection devices and methods known in the art.

### Reporter Complexes

As described above, the method may include contacting a sample with a reporter complex that includes: a first specific binding member linked to a second capture agent that specifically binds the target analyte; and a second specific binding member complementary to the first specific binding member, where the first and second specific binding members specifically bind to each other to form the reporter complex. In some cases, the second specific binding member may itself be detectable. In certain instances, the second specific binding member is linked to a detectable tag. In some cases, the first binding member is linked to both the second capture agent and a detectable tag where the detectable tag may be released by an enzymatic or chemical cleavage reaction, in some cases at a cleavage site in a cleavable linker connecting the detectable tag to the reporter complex.

In some embodiments, methods according to the present disclosure include contacting a sample with a reporter complex that includes multiple second specific binding members specifically bound to multiple first specific binding members. Reporter complexes may be linear or dendritic and may include one or more linkers linking the functional components. Suitable linkers may have a variety of structures provided they are capable of effectively positioning the various functional components of the molecules, e.g., the first specific binding members relative to the second capture agent. Suitable linkers may include, for example, polymers (e.g., PEG based polymers); alkyl groups, etc. The length of the linker may be adjusted, e.g., taking into account the size of the detectable tag to be used and/or the length and/or size of the sandwich complex to be formed (e.g., the length and/or size of the capture agent, the target analyte of interest and the specific binding members).

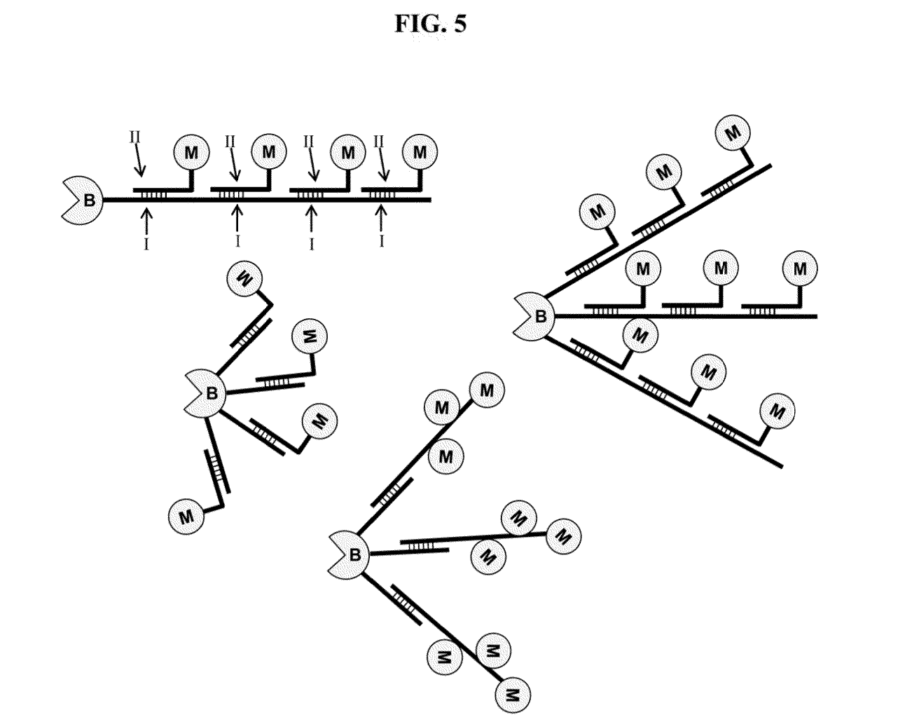

Reporter complexes that release multiple detectable tags may provide for an amplified signal and/or a lower LOD. Any convenient arrangements of multiple first and second specific binding members and detectable tags may be utilized in conjunction with a capture agent in the subject reporter complexes. In some embodiments, the reporter complex includes two or more second specific binding members specifically bound to two or more linked first specific binding members. By “linked first specific binding members” is meant that the first specific binding members are covalently or non-covalently linked to each other (e.g., directly covalently bonded or connected via an optional linker that is not a capture agent; or non-covalently linked, e.g., via a specific binding interaction). One such embodiment is depicted in FIG. 5 (top left). In some embodiments, the reporter complex includes two or more second specific binding members specifically bound to two or more non-linked first specific binding members. By “non-linked” is meant that a first specific binding member is linked to the second capture agent (e.g., directly covalently bonded or connected via an optional linker; or non-covalently linked, e.g., via a specific binding interaction) but is not linked to another first specific binding member, except indirectly via the second capture agent. One such embodiment is depicted in FIG. 5 (bottom left). In yet another embodiment, an example of which is depicted in FIG. 5 (center right), a reporter complex includes a plurality of independent sets of linked first specific binding members, wherein the independent sets are not linked to each other except indirectly via the second capture agent. In some cases, a suitable reporter complex is described by the formula B(P₁)ₙ where B is the second capture agent as described herein, P₁ is a first specific binding member, n is a suitable integer, e.g., 2 to 100, and each P₁ is specifically bound to a complementary second specific binding member P₁′. In certain embodiments of the formula B(P₁)ₙ, each P₁ is a linked first specific binding member. In certain embodiments of the formula B(P₁)ₙ, each P₁ is a non-linked first specific binding member. In some embodiments of the formula B(P₁)ₙ, one or more P₁ is a linked first specific binding member and one or more P₁ is a non-linked first specific binding member.

Reporter complexes may include a second specific binding member linked to a detectable tag. It is understood that the general formula Pₙ′-Mₙ, where Pₙ′ is the second specific binding member, Mₙ is the detectable tag and n is a suitable integer, is meant to encompass all convenient configurations of the second specific binding member linked to one or more detectable tags. In some cases, the second specific binding member is covalently linked to a detectable tag, e.g., directly covalently bonded or covalently bonded via a linker. In some instances, the second specific binding member is linked to a detectable tag via one or more intervening pairs of specific binding members (e.g., as described herein), where each pair of specific binding members is specifically bound to each other. In some instances, second specific binding member linked to a detectable tag is described by the formula:

Pₙ′-Qₙ . . . Qₙ′-Mₙ

where Pₙ′ is the second specific binding member, Mₙ is the detectable tag and Qₙ . . . Qₙ′ is a pair of specific binding members specifically bound to each other, where Pₙ′ and Qₙ and Qₙ′-Mₙ may independently be linked directly, via an optional linker, via one or more additional pairs of specific binding members, or a combination thereof. In certain embodiments, Qₙ′-Mₙ is linked via Rₙ . . . Rₙ′, a pair of specific binding members (e.g., as described herein) specifically bound to each other. In certain embodiments, Qₙ . . . Qₙ′ and/or Rₙ . . . Rₙ′ are nucleic acid duplexes.

In certain embodiments, the reporter complex includes two or more second specific binding members, each linked to one or more detectable tags. In some embodiments, the reporter complex is described by formula (I):

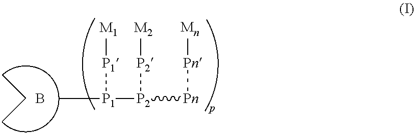

where B is the second capture agent; P₁ to Pₙ are two or more first specific binding members linked to the second capture agent B, wherein each first specific binding member may be the same or different; P₁′ to Pₙ′ are the two or more second specific binding members specifically bound to the complementary first binding member P₁ to Pₙ, wherein each second specific binding member may be the same or different; each M₁ to Mₙ is independently a detectable tag, wherein each detectable tag may be the same or different, or a mixture thereof; each n is independently a suitable integer, e.g., 2 to 100; and p is a suitable integer, e.g., 1 to 100. In some embodiments of formula (I), each first specific binding member P₁ to Pₙ is the same. In certain embodiments of formula (I), each first specific binding member P₁ to Pₙ is different. In certain instances of formula (I), each n is independently 2 to 100, such as independently 2 to 50, 2 to 20, 2 to 10, such as independently 2 to 6, such as 2, 3, 4, 5 or 6. In certain instances of formula (I), each n is 10 or more, such as 20 or more, 30 or more, 40 or more, 50 or more, 100 or more, or even more. In some instances of formula (I), each detectable tag (M₁ to Mₙ) is the same. In some instances of formula (I), each detectable tag (M₁ to Mₙ) is different. In some instances of formula (I), p is 1 to 20, such as 1 to 15, 1 to 10, e.g., 1, 2, 3, 4, 5, 6, 7, 8, 9 or 10. In certain cases of formula (I), p is 1. In certain embodiments of formula (I), p may represent an average number of appendages per second capture agent B, which may depend on the method of attachment of P₁ to B. As such, in some cases, p represents an average number per B group that is between 1 and 10, such as between 1 and 4, such as 1.2, 1.4, 1.6, 1.8, 2.0, 2.2, 2.4, 2.6, 2.8, 3.0, 3.2, 3.4, 3.6, 3.8, or 4.0.

In certain embodiments, the reporter complex is described by formula (II):

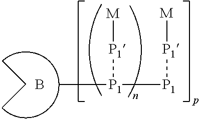

wherein B is the second capture agent; P₁ is the first specific binding member linked to the second capture agent B; P₁′ is the second specific binding member specifically bound to the complementary first binding member P₁; each M is a detectable tag; each n is independently a suitable integer, e.g., 0 to 100; and p is a suitable integer, e.g., 1 to 100. In certain instances of formula (II), each n is independently 0, 1, 2, 3, 4, 5, 6, 7, 8, 9 or 10. In certain instances, of formula (II), each n is independently 2 to 100, such as 2 to 50, 2 to 20, 2 to 10, such as 2 to 6, such as 2, 3, 4, 5 or 6. In certain instances of formula (I), each n is independently 10 or more, such as 20 or more, 30 or more, 40 or more, 50 or more, 100 or more, or even more. In certain instances, of formula (II), n is 0. In some instances of formula (II), p is 1 to 20, such as 1 to 15, 1 to 10, e.g., 1, 2, 3, 4, 5, 6, 7, 8, 9 or 10. In certain cases of formula (II), p is 1. In certain cases of formula (II), p is 2. In certain embodiments of formula (II), p may represent an average number of appendages (i.e., P₁ containing substituent chains) per second capture agent B, which may depend on the method of attachment of (P₁)ₙ₊₁ to B. As such, in some cases, p represents an average number of appendages per B group that is between 1 and 10, such as between 1 and 4, such as 1.2, 1.4, 1.6, 1.8, 2.0, 2.2, 2.4, 2.6, 2.8, 3.0, 3.2, 3.4, 3.6, 3.8, or 4.0.

In some instances, the reporter complex includes two or more second specific binding members that are specifically bound to two or more complementary sites of the first specific binding member, wherein each of the two or more second specific binding member is linked to one or more detectable tags. In such reporter complexes, the two or more second specific binding members may each be released from the complex using one or more displacement binding members. In certain embodiments, each of the two or more second specific binding members is the same and may be released using one type of displacement binding member, e.g. a plurality of nucleic acid displacement binding members having the same sequence. In certain embodiments, each of the two or more second specific binding members is different and may be released using two or more displacement binding members. In some instances, the method further includes detecting the detectable tags linked to the displaced second specific binding members.

Linkers of interest that find use in the subject compositions include polymeric linker moieties. Examples of such include poly(ethyleneimine) and poly(alkyleneoxide) linker moieties, such as poly(methylene oxide) (i.e., (—OCH₂—)ₙ), poly(ethylene oxide) (i.e., (—OCH₂CH₂—)ₙ), polypropylene oxide) (i.e., (—OCH₂CH(CH₃)—)ₙ), or the like. Other examples of polymeric linking moieties include poly(amino acid)s, poly(saccharide)s, and poly(nucleic acid)s. Where the linker is a polymeric linking moiety, the molecular weight of the linker may be between about 100 Da and about 100,000 Da, or between about 100 Da and 10,000 Da, or between about 100 Da and 1,000 Da. In some embodiments, the linker has block-like character. For example, L¹ may include two, three, four, or more blocks of any size of the above-mentioned example polymeric moieties. In other embodiments, L¹ is not a polymeric moiety in the sense that it does not have an identifiable repeating moiety. Examples of such L¹ groups include an amino acid (e.g., tyrosine). For example, linking moieties containing a moiety selected from alkyl groups, amide groups, ether groups, ester groups, and combinations thereof. Examples include —(CH₂)ₙ—C(═O)—NH—, —(OCH₂CH₂)ₙ—C(═O)—NH—, —C(═O)—NH—CH₂CH₂—(OCH₂CH₂)ₙ—C(═O)—NH—, —C(═O)—NH—(CH₂)ₙ—C(═O)—NH—, and the like, wherein n is an integer equal to or greater than 0.

It should be noted that the reporter complexes described above and elsewhere herein may be formed in a variety of ways and via any number of suitable steps in any suitable order. For example, a sample may be contacted with a reporter complex that includes each of the first specific binding member, the second capture agent, the second specific binding member, and optionally a detectable label or labels, bound as a complex. Alternatively, a sample may be contacted with one or more of the above components independently, and the reporter complex may be formed as a result of the addition of all the components. For example, the second specific binding member may be added subsequently to the formation of a sandwich complex including the target analyte, the first capture agent, and the second capture agent linked to the first specific binding member.

Any suitable order and combination of the above components may be utilized to form the reporter complexes and sandwich complexes of the present disclosure. In some embodiments, one or more of the first capture agent, the first specific binding member, the second capture agent, the second specific binding member, and the detectable tag may be added independently to the sample to form the sandwich complex comprising the reporter complex.

The above components find use in methods for detecting the presence, absence and/or amount of a target molecule or other analyte of interest in a sample.

### Methods

As summarized above, the present disclosure provides methods of detecting a target analyte in a sample. In some embodiments, such methods include:

(a) contacting the sample with:
(i) a first capture agent that specifically binds a target analyte; and
(ii) a reporter complex, comprising:
   (A) a first specific binding member linked to a second capture agent that specifically binds the target analyte;
   (B) a second specific binding member complementary to the first specific binding member, wherein the first and second specific binding members specifically bind to each other to form the reporter complex;
under conditions sufficient to specifically bind the first and second capture agents to the target analyte to produce a sandwich complex;

(b) separating the sandwich complex from the sample; and

(c) releasing the second specific binding member from the sandwich complex.

In some embodiments, the subject methods include releasing (e.g., via displacement or cleavage, as described herein) a detectable tag from the reporter complex. The releasing step may be achieved using any convenient methods. In certain embodiments of the method, the releasing step (c) is achieved using a displacement binding member that is complementary to the first or second specific binding member. In some embodiments of the method, the displacement binding member is complementary to the first specific binding member and step (c) includes specifically binding the displacement binding member and the first specific binding member. In certain embodiments, step (c) includes hybridizing the displacement binding member and the first specific binding member, thereby releasing the second specific binding member from the reporter complex. In some embodiments, the displacement binding member is complementary to the second specific binding member and step (c) includes specifically binding the displacement binding member and the second specific binding member. In certain embodiments, step (c) includes hybridizing the displacement binding member and the second specific binding member, thereby releasing the second specific binding member from the reporter complex.

In some instances, second specific binding member includes a detectable tag (e.g., as described herein) and the method further includes detecting the detectable tag. Detecting the detectable tag may be achieved using any convenient methods. Methods of analyzing a target of interest that may be adapted for use in the subject methods, include but are not limited to, flow cytometry, in-situ hybridization, enzyme-linked immunosorbent assays (ELISAs), western blot analysis, magnetic cell separation assays, fluorochrome purification chromatography, fluorescence spectroscopy, nucleic acid sequencing, fluorescence in-situ hybridization (FISH), protein mass spectroscopy and flow cytometry. Detection may be achieved directly via a reporter molecule, or indirectly by a secondary detection system. The latter may be based on any one or a combination of several different principles including but not limited to, antibody labelled anti-species antibody and other forms of immunological or non-immunological bridging and signal amplification systems (e.g., biotin-streptavidin technology, protein-A and protein-G mediated technology, or nucleic acid probe/anti-nucleic acid probes, and the like).

In certain embodiments of the method, the releasing step (c) is achieved by cleaving the detectable tag from the reporter complex. In such cases, the reporter complex may include a cleavable group (e.g., as described herein).

In some cases, releasing the detectable tag is achieved using a biocompatible aqueous eluent that may include one or more components such as a displacement binding member. In some cases, the detectable tag is released under conditions in which target analyte biological activity and/or viability is preserved. As such, the disclosure provides a biocompatible aqueous buffer or eluent for washing and/or eluting materials from the support. As used herein, the term “biocompatible” refers to an aqueous eluent that preserves the functional and/or structural integrity of biomolecules of interest, e.g., in the sample or components of the sandwich complex. By functional and/or structural integrity of biomolecules is meant that a function of interest and/or a structural element of interest (e.g., a primary, secondary and/or tertiary structural element) of the biomolecule is preserved such that any convenient downstream analysis and/or detection (e.g., direct or indirect) steps of the method are preserved. In some embodiments, a biocompatible eluent or buffer refers to an aqueous solution that is non-cytotoxic and non-denaturing to biomolecules of interest. In addition, the components of the biocompatible aqueous eluent may be selected such that the eluent has no adverse effects on subsequent analysis and/or use of the target analytes. In some embodiments, the biocompatible aqueous eluent includes a binding competitor or inhibitor that is capable of disrupting the specific binding of a binding member of interest. By disrupting the specific binding is meant that two binding members of interest may be more easily disassociated. The binding competitor or inhibitor may have any convenient affinity for one of the binding members. In some cases, the binding competitor or inhibitor binds with a relatively low affinity, but may disrupt specific binding at a sufficient and desirable concentration in the eluent. It is understood that the biocompatible aqueous eluent may further include a variety of components in conjunction with the binding competitor or inhibitor to promote dissociation.

The biocompatible buffer may be utilized in any convenient steps of the method, such as binding steps, blocking steps, washing steps and/or eluting or displacement steps. In some instances, the biocompatible buffer preserves the structure and/or activity of a molecule of interest, e.g., an enzymatic activity or a binding activity. In some cases, the biocompatible buffer preserves the structural integrity of a molecule of interest, e.g., the primary, secondary and/or tertiary structure of the molecule is preserved such that the biocompatible buffer doesn't interfere with any downstream steps, e.g., analysis, separation and/or detection steps. Any convenient buffers and buffer components may find use in the biocompatible buffers. For example, PBS for example, (aqueous solution buffer with salt from 1 mm to 1M for example), or HEPES, having a pH between 5 and 10. In some cases, a biocompatible buffer does not include an organic solvent.

Any convenient method may be used to contact the sample with a capture agent and a reporter complex that specifically bind to the target analyte. In some instances, the sample is contacted with the subject composition under conditions in which the first and second capture agents specifically binds to the target analyte, if present, to produce a sandwich complex.

For specific binding of the first and second capture agents with the target analyte, an appropriate solution may be used that maintains the structure and/or biological activity of the target and/or the sample. The solution may be a balanced salt solution, e.g., normal saline, PBS, Hank's balanced salt solution, etc., conveniently supplemented with fetal calf serum, human platelet lysate or other factors, in conjunction with an acceptable buffer at a suitable concentration, such as from 5-25 mM or 25-300 mM. Convenient buffers include HEPES, phosphate buffers, lactate buffers, etc. Various media are commercially available and may be used according to the nature of the target cells, including dMEM, HBSS, dPBS, RPMI, Iscove's medium, etc., frequently supplemented with fetal calf serum or human platelet lysate. The final components of the solution may be selected depending on the components of the sample which are included. The sample may include a heterogeneous cell population from which target analytes are isolated. In some instances, the sample includes peripheral whole blood, peripheral whole blood in which erythrocytes have been lysed prior to cell isolation, cord blood, bone marrow, density gradient-purified peripheral blood mononuclear cells or homogenized tissue. In some cases, the sample includes hematopoetic progenitor cells (e.g., CD34+ cells) in whole blood, bone marrow or cord blood. In certain embodiments, the sample includes tumor cells in peripheral blood. In certain instances, the sample is a sample including (or suspected of including) viral particles (e.g., HIV).

The temperature at which specific binding of the first and second capture agents with the target analyte takes place may vary, and in some instances may range from 5° C. to 50° C., such as from 10° C. to 40° C., 15° C. to 40° C., 20° C. to 40° c., e.g., 20° C., 25° C., 30° C., 35° C. or 37° C. (e.g., as described above). In some instances, the temperature at which specific binding takes place is selected to be compatible with the biological activity or viability of the target analyte and/or other components of the sample. In certain instances, the temperature is 25° C., 30° C., 35° C. or 37° C. In certain cases, one or more of the target analyte and capture agents is an antibody or fragment thereof and the temperature at which specific binding takes place is room temperature (e.g., 25° C., 30° C., 35° C. or 37° C.). Any convenient incubation time for specific binding may be selected to allow for the formation of a desirable amount of sandwich complex, and in some instances, may be 1 minute (min) or more, such as 2 min or more, 10 min or more, 30 min or more, 1 hour or more, 2 hours or more, or even 6 hours or more.

The subject methods may further include one or more optional washing steps. In some cases, the washing steps are used to remove unbound material of the sample from a support bound sandwich complex. In certain cases, the washing steps are used to remove unbound and/or excess agents or components of a complex from a support bound sandwich complex, such as excess reporter complex or a component of the reporter complex. For example, in some instances of the method excess reporter complex or components of the same are contacted with a sample of interest in order to produce a sandwich complex. In such cases, excess reporter complex and/or components thereof are present in the contacted sample, which may be removed by one or more wash steps. In certain cases, the support is a cell. Any convenient separation and/or washing methods may be used, e.g., washing the immobilized support with a biocompatible buffer which preserves the specific binding interactions of the sandwich complex and the reporter complex. Separation and optional washing of unbound material of the sample from the support provides for an enriched population of target analytes where undesired cells and material may be removed.

In certain embodiments, the method further includes detecting the detectable tag (e.g., as described herein). Any convenient methods may be utilized to detect and/or analyze the target analyte via the detectable tag in conjunction with the subject methods and compositions. Methods of detecting and/or analyzing that find use in the subject methods, include but are not limited to, flow cytometry methods, in-situ hybridization, enzyme-linked immunosorbent assays (ELISAs), western blot analysis, magnetic cell separation assays, fluorescence spectroscopy, nucleic acid sequencing, fluorescence in-situ hybridization (FISH) and protein mass spectroscopy. Detection may be achieved directly via a reporter molecule, or indirectly by a secondary detection system. The latter may be based on any one or a combination of several different principles including but not limited to, antibody labelled anti-species antibody and other forms of immunological or non-immunological bridging and signal amplification systems (e.g., biotin-streptavidin technology, protein-A and protein-G mediated technology, or nucleic acid probe/anti-nucleic acid probes, and the like). The label used for direct or indirect detection may be any detectable reported molecule.

Detecting the detectable tag may include exciting a fluorescent dye with one or more lasers, and subsequently detecting fluorescence emission from the dye using one or more optical detectors. In some embodiments, the methods further include counting, sorting, or counting and sorting a labeled particle. The detectable tags may be detected and uniquely identified by exposing them to excitation light and measuring the fluorescence produced in one or more detection channels, as desired. The excitation light may be from one or more light sources and may be either narrow or broadband. Examples of excitation light sources include lasers, light emitting diodes, and arc lamps. Fluorescence emitted in detection channels used to identify the detectable tags and components associated therewith may be measured following excitation with a single light source, or may be measured separately following excitation with distinct light sources. If separate excitation light sources are used to excite the detectable tags, the tags may be selected such that all the tags are excitable by each of the excitation light sources used.

As discussed elsewhere herein, the reporter complexes and sandwich complexes of the present disclosure may be formed in a variety of ways and via any number of suitable steps in any suitable order. For example, a sample may be contacted with a reporter complex that includes each of the first specific binding member, the second capture agent, the second specific binding member, and optionally a detectable label or labels, bound as a complex. Alternatively, a sample may be contacted with one or more of the above components independently, and the reporter complex may be formed as a result of the addition of all the components. For example, the second specific binding member may be added subsequently to the formation of a sandwich complex including the target analyte, the first capture agent, and the second capture agent linked to the first specific binding member.

Any suitable order and combination of the above components, including the sample, may be utilized to form the reporter complexes and sandwich complexes of the present disclosure. In some embodiments, one or more of the first capture agent, the first specific binding member, the second capture agent, the second specific binding member, and the detectable tag may be added independently to the sample to form the sandwich complex comprising the reporter complex.

Methods Including Cleaving the Detectable Tag from the Reporter Complex

As described above, aspects of the method include releasing the detectable tag from the reporter complex by cleavage (e.g., chemical, enzymatic or photocleavage). Cleavage may include cleavage of a cleavable group in the first and/or second specific binding member to result in release of the second specific binding member (or a fragment thereof) from the reporter complex. As described herein, in some cases, the second specific binding member may itself be detectable or it may further include a detectable tag. A cleavable group may be included in the reporter complex at any convenient location to provide for selective cleavage of the detectable tag from the reporter complex upon application of a stimulus. Application of a stimulus may include contacting the reporter complex with an enzyme or a chemical agent, or irradiation with light (e.g., of a particular wavelength) or any other suitable stimulus that would cause cleavage.

As such, the reporter complex may include a cleavable group that links the detectable moiety to the capture agent. A variety of cleavable groups may be utilized in the subject reporter complexes and methods to provide for release of a detectable tag from the portion of the reporter complex that includes the sandwich complex with the target analyte. Cleavable linkers that include cleavable groups of interest, include but are not limited to those cleavable linkers as described in Olejnik et al. (Methods in Enzymology 1998 291:135-154), and further described in U.S. Pat. No. 6,027,890; Olejnik et al. (Proc. Natl. Acad Sci, 92:7590-94); Ogata et al. (Anal. Chem. 2002 74:4702-4708); Bai et al. (Nucl. Acids Res. 2004 32:535-541); Zhao et al. (Anal. Chem. 2002 74:4259-4268); and Sanford et al. (Chem. Mater. 1998 10:1510-20). Cleavable linkers that may be employed in the subject reporter complexes include, but are not limited to, electrophilically cleavable linkers, nucleophilically cleavable linkers, photocleavable linkers, metal cleavable linkers, electrolytically-cleavable, enzymatically cleavable linkers, and linkers that are cleavable under reductive and oxidative conditions.

A cleavable group may include a substrate for an enzyme, such as a restriction enzyme or a protease. As such, the reporter complex may include a cleavable group including a nucleic acid sequence or a peptidic sequence that is capable of being selectively cleaved by an enzyme of interest. In certain embodiments, the second specific binding member includes the cleavable group. In certain embodiments, where the first and second specific binding members are nucleic acids and specifically bind via hybridization, the resulting duplex may include a restriction site for a restriction enzyme of interest. The first and second specific binding members may be selected to provide for an enzyme cleavage site (e.g., a cleavable group).

As such, in some embodiments, a method of detecting a target analyte in a sample includes: (a) contacting the sample with:
(i) a first capture agent that specifically binds a target analyte; and
(ii) a reporter complex, comprising:
   (A) a first specific binding member linked to a second capture agent that specifically binds the target analyte;
   (B) a second specific binding member linked to a detectable tag,
wherein the second specific binding member is complementary to the first specific binding member and the first and second specific binding members are specifically bound to form the reporter complex;

under conditions sufficient to specifically bind the first and second capture agents to the target analyte to produce a sandwich complex;

(b) separating the sandwich complex from the sample; and

(c) cleaving the detectable tag from the sandwich complex.

In certain embodiments of the method, step (c) includes contacting the sandwich complex with an enzyme under conditions sufficient to cleave a portion of the reporter complex that links the detectable tag to the second capture agent. In certain instances, the cleaving step releases a portion of the second specific binding member in addition to the detectable tag. In certain instances, the cleaving releases portions of both the first and second specific binding members in addition to the detectable tag. In some cases, cleaving the detectable tag is achieved using a biocompatible aqueous eluent that may include one or more components such as an enzyme or other cleavage agent. In certain instances, the reporter complex further includes a cleavable linker that links the detectable tag and the second capture agent and step (c) comprises applying a stimulus (e.g., a cleaving reagent, light) to cleave the cleavable linker and release the detectable tag. In some instances of the method, the method further includes detecting the detectable tag. In some cases, in step (c), the detectable tag is released into a solution having a volume that is 50% or less the volume of the sample. In some embodiments of the method, the target analyte is a nucleic acid, a protein, a hormone, a lipid or a sugar. In certain instances of the method, the first capture agent and the second capture agent are independently selected from a nucleic acid, a protein, a peptide, or a small molecule (e.g., an antibody, a hapten, an aptamer, etc). In some instances of the method, the first capture agent is linked to a support (e.g., a bead, a particle (e.g., a magnetic particle), a gel, a membrane, a fiber, a biosensor chip surface, a vessel (e.g., a tube surface), cell, or a bacterium). In some embodiments of the method, the target analyte is a target protein and the first capture agent and the second capture agent are each an antibody, an antibody fragment or a derivatized antibody. In certain embodiments of the method, the first specific binding member and the second specific binding member are independently selected from a nucleic acid or nucleic acid analog (e.g., a DNA, a RNA or a PNA). In certain instances of the method, the detectable tag is an enzyme, a nucleic acid, a polypeptide, a particle, an affinity tag, a fluorophore, a chromophore, a luminescent tag, a radioactive tag or a chemoselective tag.

In some embodiments, a method of detecting a target analyte in a sample includes:

(a) contacting the sample with:
(i) a first capture agent that specifically binds a target analyte; and
(ii) a reporter molecule, comprising:
   a first specific binding member linked to a second capture agent that specifically binds the target analyte; and
   a detectable tag;

under conditions sufficient to specifically bind the first and second capture agents to the target analyte to produce a sandwich complex;

(b) separating the sandwich complex from the sample; and

(c) cleaving the detectable tag from the sandwich complex.

In certain embodiments, the reporter complex further includes a second specific binding member, wherein the second specific binding member is complementary to the first specific binding member and the first and second specific binding members are specifically bound to form the reporter molecule. The detectable tag may be linked to the first specific binding member. The detectable tag may be linked to the second specific binding member. In certain instances, the cleaving step releases a portion of the first specific binding member in addition to the detectable tag. In certain instances, the cleaving releases portions of both the first and second specific binding members in addition to the detectable tag.

Also provided are methods that include indirect detection of low levels of target analyte facilitated by release of the second specific binding member from the reporter complex into a reduced volume of liquid relative to the volume of liquid in the sample. As such, the method may be utilized to concentrate the detectable moiety relative to the target analyte to facilitate detection. In some embodiments of the method, in step (c), the second specific binding member is released into a solution having a volume that is 50% or less than the volume of the sample, such as 40% or less, 30% or less, 20% or less, 10% or less, 5% or less, 2% or less, 1% or less, or even 0.1% or less than the volume of the sample. In certain embodiments of the method, the limit of detection (LOD) of the target analyte as measured indirectly via detection of the detectable moiety is 2-fold lower or more than the LOD of a directly detected target analyte, such as 5-fold lower or more, 10-fold lower or more, 20-fold lower or more, 50-fold lower or more, 100-fold lower or more, or even 1000-fold lower or more. In some cases, by directly detected is meant detected by spectroscopy, e.g., fluorescence, UV spectroscopy or mass spectroscopy.

A variety of methods may be utilized to facilitate detection of an analyte concentration below the LOD of a particular detection method. For example, analyte from a larger sample can be concentrated to produce a smaller sample with an analyte concentration equal to or above the LOD for a particular method. Alternatively, a reporter system such as that depicted in FIG. 5 can be utilized, where for each analyte molecule, 2 or more detectable tags are released. Thus the tag or “reporter analyte” concentration will be higher than the original analyte of interest. The tag concentration will be within the limits of detection of the detection method. A combination approach may also be utilized in which a multi-tag reporter system is utilized, and the tags are released into a smaller volume to further increase their concentration.

Aspects of the subject methods include analysis of a sample that includes (or is suspected of including) two or more target analytes. In some embodiments, the method includes: contacting the sample with two or more distinct reporter complexes (e.g., as described herein) that each further include a distinct addressable tag to produce two or more distinct sandwich complexes; and releasing second specific binding members from the two or more distinct sandwich complexes using at least one displacement binding member, wherein the second specific binding members are linked to the distinct addressable tags. In certain embodiments, the method further includes capturing the distinct addressable tags on a support, such as a microarray or an encoded bead support that is capable of capturing the distinct addressable tags (e.g., by specifically binding or chemoselectively linking to the tags). Any convenient multiplexing technology or library encoding technology may be adapted for use in connection with the addressable tags of the subject compositions and methods. In some embodiments, by “addressable” is meant a tag that is spatially addressable, e.g., provides for separation of the tag-containing moiety from a mixture, e.g., by binding to a particular location on a microarray or microbead. Addressable tags of interest include, but are not limited, nucleic acids, haptens, antigens, antibodies and a specific binding member.

Alternatively, or in addition, to the use of an addressable tag as described above, multiplexing can be achieved through the use of distinct detectable tags. For example, in some embodiments, the method includes: contacting the sample with two or more distinct reporter complexes (e.g., as described herein) that each further include a distinct detectable tag (or combination of detectable tags) to produce two or more distinct sandwich complexes; and releasing the distinct detectable tags (or combination of detectable tags), from the two or more distinct sandwich complexes, e.g., by releasing second specific binding members comprising or linked to the detectable tags, using at least one displacement binding member. Distinct fluorophores which emit light at different wavelengths, or any other suitable distinguishably detectable tag, can be utilized in such embodiments facilitating the detection of different analytes from the same sample with or without physical separation.

In some instances of the method, each reporter complex includes a distinct second specific binding member, and the method includes releasing the distinct second specific binding members using two or more distinct displacement binding members, e.g., two or more nucleic acid displacement binding members having different sequences. The two or more distinct second binding members may be released using any convenient methods. In some cases, the two or more distinct second binding members are released simultaneously. In some cases, the two or more distinct second binding members are released sequentially, e.g., individually or in batches. The identity and arrangement of the two or more distinct second binding members may be selected as needed to provide for any convenient degree of complexity in the subject multiplexed methods. In some embodiments of the method, each reporter complex includes a second specific binding member complementary to the same first specific binding member and the method includes releasing all of the second specific binding members using a single type of displacement binding member, e.g., two or more nucleic acid displacement binding members have the same sequence. In certain embodiments of the method, the displacement binding member is selected from polypeptides, nucleic acids and small molecules. In certain cases, the displacement binding member is a nucleic acid.

In some embodiments where the sample comprises two or more target analytes, the method includes:
(a) contacting the sample with: two or more distinct first capture agents that specifically bind different target analytes; and two or more distinct reporter complexes, each including a distinct second capture agent that specifically binds one of the different target analytes; where the two or more distinct reporter complexes each further include a distinct addressable tag and a detectable tag linked to (or comprised by) the second specific binding member; under conditions sufficient to specifically bind each of the two or more distinct first capture agents and the corresponding two or more distinct reporter complexes to different target analytes to produce two or more distinct sandwich complexes;
(b) separating the two or more distinct sandwich complexes from the sample;
(c) releasing the second specific binding members from the two or more distinct sandwich complexes using at least one displacement binding member; and
(d) capturing two or more distinct addressable tags on a support.

In certain embodiments of the method, each of the two or more distinct reporter complexes include a distinct second specific binding member and step (c) includes releasing the distinct second specific binding members using two or more distinct displacement binding members, e.g., two or more nucleic acid displacement binding members have different sequences. In certain instances, the two or more distinct displacement binding members are independently selected from polypeptides, nucleic acids and small molecules. In some cases, the two or more distinct displacement binding members are nucleic acids.

In some instances of the method, each of the two or more distinct reporter complexes includes a second specific binding member complementary to the same first specific binding member and step (c) comprises releasing all of the second specific binding members using a single type of displacement binding member, e.g., two or more nucleic acid displacement binding members have the same sequence. In certain instance, the displacement binding member is a nucleic acid.

In some embodiments where the sample comprises two or more target analytes, the method includes:
(a) contacting the sample with: two or more distinct first capture agents that specifically bind different target analytes; and two or more distinct reporter complexes, each including a distinct second capture agent that specifically binds one of the different target analytes; where the two or more distinct reporter complexes each further include a distinct detectable tag linked to (or comprised by) the second specific binding member; under conditions sufficient to specifically bind each of the two or more distinct first capture agents and the corresponding two or more distinct reporter complexes to different target analytes to produce two or more distinct sandwich complexes;
(b) separating the two or more distinct sandwich complexes from the sample;
(c) releasing the second specific binding members from the two or more distinct sandwich complexes using at least one displacement binding member; and
(d) detecting the two or more distinct detectable tags.

In certain embodiments of the method, each of the two or more distinct reporter complexes include a distinct second specific binding member and step (c) includes releasing the distinct second specific binding members using two or more distinct displacement binding members, e.g., two or more nucleic acid displacement binding members have different sequences. In certain instances, the two or more distinct displacement binding members are independently selected from polypeptides, nucleic acids and small molecules. In some cases, the two or more distinct displacement binding members are nucleic acids.

In some instances of the method, each of the two or more distinct reporter complexes includes a second specific binding member complementary to the same first specific binding member and step (c) comprises releasing all of the second specific binding members using a single type of displacement binding member, e.g., two or more nucleic acid displacement binding members have the same sequence. In certain instance, the displacement binding member is a nucleic acid.

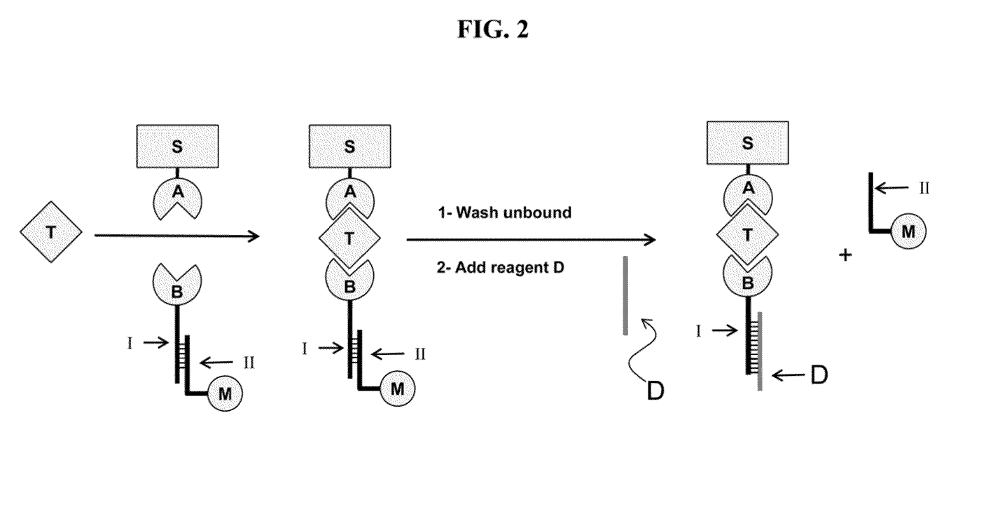

One embodiment of a detection method according to the present disclosure is depicted generally in FIG. 2. A sample containing a target analyte of interest “T” is contacted with reporter complex R (e.g., as described in FIG. 1) and the first capture agent “A” which can be optionally tethered to a support S. After a suitable incubation period, T becomes simultaneously bound to A and B resulting in a sandwich complex A:T:B. Subsequently: 1) the sandwich complex is separated from the remaining sample and any unbound materials are washed away; 2) complex ATB is further contacted with displacement binding member “D” which binds to I by hybridization of complementary sequences causing displacement of II (e.g., as described in FIG. 2) into the supernatant, 3) II or M are measured directly or indirectly from the supernatant using any convenient methods. The stringency of the reaction conditions may be adjusted and one or more wash steps may be utilized to minimize non-specific binding of the target analyte or other components of the method. This can be accomplished by adjusting, for example, the pH, temperature and/or salt concentration of the wash conditions.

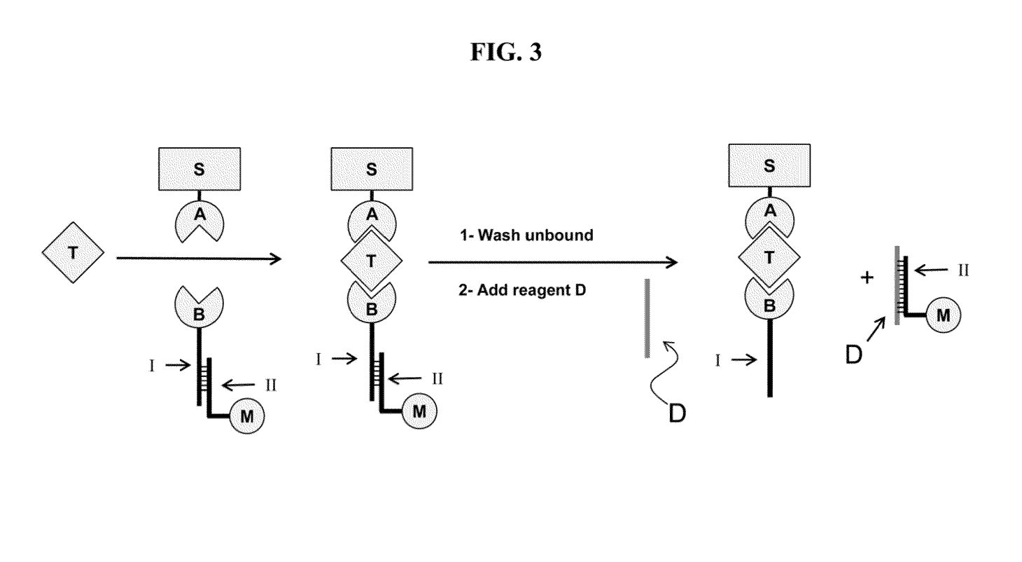

Another embodiment of the invention is depicted in FIG. 3, where further to the embodiment described in FIGS. 1-2, displacement binding member “D” binds to II by hybridization of complementary sequences causing displacement of II into the supernatant in the form of a complex with D, and II or M are measured directly or indirectly from the supernatant using any convenient methods.

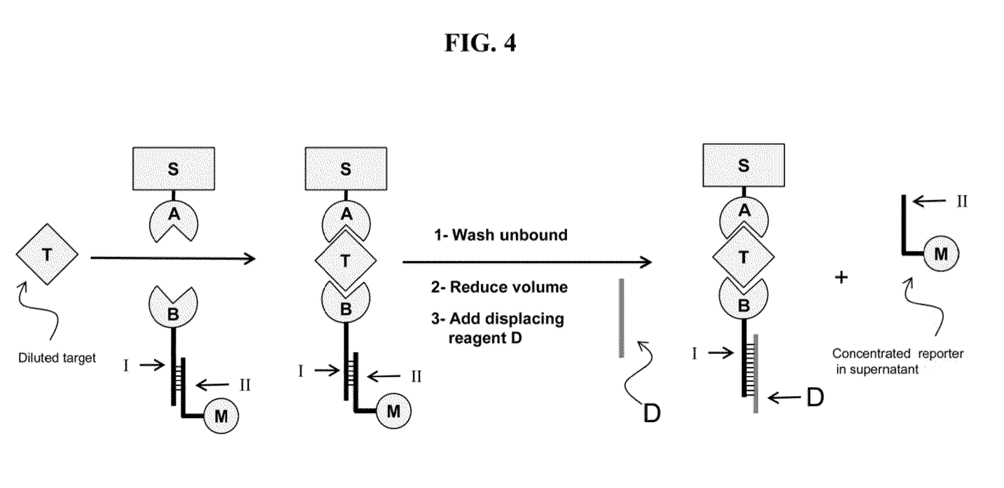

One embodiment of a detection method according to the present disclosure is depicted generally in FIG. 4. Further to the embodiments described in FIGS. 1-3, target analyte T is present in a large sample volume, e.g., between 0.1 and 10 ml, and the washed sandwich complex A:T:B is exposed to reagent D in a smaller volume, e.g., between 0.005 to 0.050 mL, causing II (see FIG. 1) to be displaced into the smaller volume and be substantially more concentrated than T was in the original sample.

One embodiment of a detection method according to the present disclosure is depicted generally in FIG. 5. Further to the embodiments described in FIGS. 1-4, reporter complex “R” includes multiple copies of strand “II” some examples of which are described in FIG. 5. Thus, when a single type of displacement binding member “D” is added, e.g., two or more nucleic acid displacement binding members have the same sequence, multiple copies of “II” are released per each A:T:B sandwich complex.

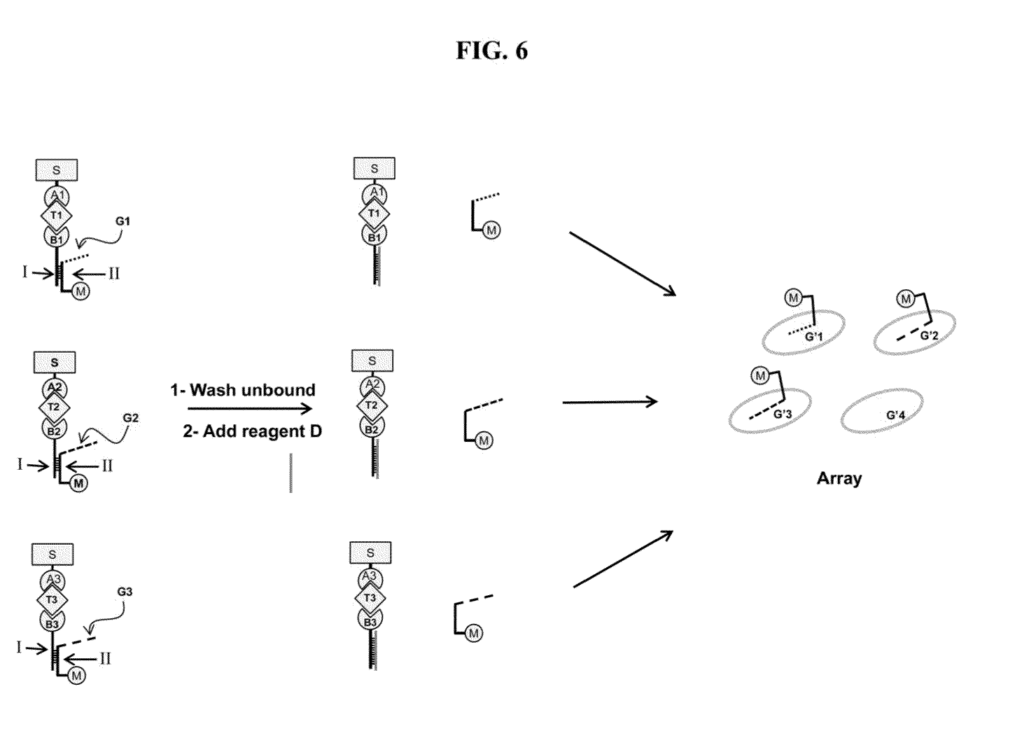

One embodiment of a detection method according to the present disclosure is depicted generally in FIG. 6. Further to the embodiments described in FIGS. 1-5, strand “II” (e.g., as described in FIG. 1) further includes a unique addressable tag “G” tethered to it, and multiple versions of the reporter complex “R” are prepared such that there is a correspondence between the specific target binding agent “B” and the unique addressable tag “G”. For example, R1 contains B1 and G1; R2 contains B2 and G2, R3 contains B3 and G3, and specifically recognize target analytes T1, T2, and T3, respectively. In this embodiment of the method, multiple target analytes are simultaneously detected from the same sample by contacting the sample with a mixture of reporter complexes (e.g., R1, R2, R3, etc.) and a mixture of corresponding first capture agents (e.g., A1, A2, A3, etc.) to provide for formation of multiple sandwich complexes (e.g., A1 T1B1, A2T2B2, A3T3B3, etc.). After washing unbound sample material from the separated sandwich complexes and adding a single universal displacement binding member “D”, corresponding versions of strand “II” (see e.g., FIG. 1) with a linked detectable tag “M” and a unique addressable tag G1, G2, or G3 are released into solution. Thus, the amount of each target analyte can be simultaneously quantified by measuring the measurable moiety “M” associated with each addressable tag “G” using any convenient liquid or solid array formats, where the elements of the array contain corresponding array capture agents G1′, G2′ and G3′ to specifically recognize the G1, G2, and G3 addressable tags respectively.

One or more steps of the methods described herein, e.g., one or more binding, washing, displacement, or detection steps may be performed under physiological conditions. “Physiological conditions” as use herein refer to conditions which are sufficient to preserve the integrity and functionality of one or more biological components involved in the performance of the methods. Depending on the specific biological components, e.g., biomolecules, and reagents utilized in the performance of the methods, the range of physiological conditions may vary greatly. Exemplary conditions which may be utilized in some embodiments of the disclosed methods are as follows: pH of from about 4 to about 10, e.g., from about 5 to about 9, or from about 6 to about 8, e.g., 7; and/or temperature of from about about 2° C. to about 75° C., e.g., from about 4° C. to about 65° C., from about 10° C. to about 55° C., from about 15° C. to about 45° C., from about 20° C. to about 40° C., or from about 25° C. to about 37° C.; and/or salt concentration of from about 0 M to about 3 M, e.g., from about 0.01 M to about 1M, or from about 0.01 M to about 0.5 M. Any suitable buffer may be utilized in one or more of the steps of the methods described herein, including, but not limited to, Tris buffers, MES buffers, HEPES buffers, Tricine buffers, Carbonate buffers, Acetate buffers, Borate buffers, phosphate buffers, and the like.

### Systems

Aspects of the invention further include systems for use in practicing the subject methods. A subject analysis system may include an electrophoresis device. Any convenient electrophoresis devices may be utilized in the subject systems. One such device is described in U.S. Pat. No. 8,263,022, the disclosure of which is incorporated by reference herein in its entirety and for all purposes. The electrophoresis device may include an analyte detection zone that includes; a first capture agent that specifically binds a target analyte (e.g., as described herein); and a reporter complex (e.g., as described herein), including: a first specific binding member linked to a second capture agent that specifically binds the target analyte; and a second specific binding member complementary to the first specific binding member and the first and second specific binding members are specifically bound to form the reporter complex. In some embodiments of the system, the second specific binding member is linked to a detectable tag. In certain embodiments of the system, the second specific binding member comprises a detectable tag. In some instances of the system, the analyte detection zone further includes a displacement binding member that is complementary to the first or second specific binding member.

The electrophoresis device may further include an analyte concentration zone. The concentration zone is a zone of the device where the detectable moiety may be released (e.g., via displacement or cleavage, as described herein) from the reporter complex into a desired volume of liquid. As such, in some cases, the system includes a solution of the detectable moiety at a relatively higher concentration relative to the initial concentration of the target analyte in the sample.

In some embodiments, the system further includes computer-based systems configured to receive, store and/or analyze data collected from one or more components of the system (e.g., a detector). In certain embodiments, the system further includes computer-based systems configured to detect the presence of the fluorescent signal. A “computer-based system” refers to the hardware means, software means, and data storage means used to analyze the information of the present invention. The hardware of the computer-based systems of the present disclosure generally includes a central processing unit (CPU), input means, output means, and data storage means. A skilled artisan can readily appreciate that any one of the currently available computer-based system are suitable for use in the present system. The data storage means may include any manufacture including a recording of the present information as described above, or a memory access means that can access such a manufacture.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g., word processing text file, database format, etc.

A “processor” references any hardware and/or software combination that will perform the functions required of it. For example, any processor herein may be a programmable digital microprocessor such as available in the form of an electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

In addition to the sensor device and signal processing module, e.g., as described above, systems of the invention may include a number of additional components, such as data output devices, e.g., monitors and/or speakers, data input devices, e.g., interface ports, keyboards, etc., fluid handling components, power sources, etc.

### Compositions

Aspects of the invention further include compositions for use in practicing the subject methods. The compositions of the invention can be provided for use in, for example, the methodologies described above. The subject compositions may include one or more of any of the components (e.g., as described herein) of the subject methods, such as first capture agents, optionally linked to a support, reporter complexes, detectable tags, displacement binding members, addressable tags and corresponding array supports, etc.

In some embodiments, the composition includes a reporter complex (e.g., as described herein). In some embodiments, the composition includes: (a) a first capture agent that specifically binds a target analyte; and (b) a reporter complex (e.g., as described above). The reporter complex may include: a first specific binding member linked to a second capture agent that specifically binds the target analyte; and a second specific binding member complementary to the first specific binding member and the first and second specific binding members are specifically bound to form the reporter complex. In certain embodiments of the composition, the second specific binding member is linked to a detectable tag. In certain embodiments of the composition, the second specific binding member includes a detectable tag. In certain embodiments of the composition, the composition further includes the target analyte. In certain embodiments of the composition, the second specific binding member is further linked to an addressable tag. In certain embodiments of the composition, the reporter complex includes two or more second specific binding members that are hybridized to two or more complementary sites of the first specific binding member, wherein each of the two or more second specific binding members is linked to a detectable tag. In certain embodiments of the composition, the first capture agent is linked to a support. In certain embodiments of the composition, the composition further includes a displacement binding member that is complementary to the first or second specific binding member.

In some embodiments, the composition includes a (e.g., as described herein); (a) a reporter complex, including: a first specific binding member linked to a capture agent that specifically binds a target analyte; and a second specific binding member complementary to the first specific binding member and the first and second specific binding members are specifically bound to form the reporter complex; and (b) a displacement binding member that is complementary to the first or second specific binding member. In some embodiments of the composition, the second specific binding member is linked to a detectable tag. In certain embodiments of the composition, the second specific binding member comprises a detectable tag. In some embodiments of the composition, the displacement binding member is complementary to the first specific binding member. In some embodiments of the composition, the displacement binding member is complementary to the second specific binding member. In certain embodiments of the composition, the second specific binding member is further linked to an addressable tag. In some embodiments of the composition, the reporter complex includes two or more second specific binding members that are specifically bound to two or more complementary sites of the first specific binding member, wherein each of the two or more second specific binding members is linked to a detectable tag. In some embodiments of the composition, the composition further includes a second capture agent that specifically binds the target analyte. In some embodiments of the composition, the second capture agent is linked to a support. In some embodiments of the composition, the composition further includes the target analyte. In some embodiments of the composition, the reporter complex includes a cleavable linker that links the detectable tag to the reporter complex, such as a cleavable linker including a cleavage site that is susceptible to cleavage by an enzymatic or chemical cleavage reagent.

### Kits

Aspects of the invention further include kits for use in practicing the subject methods and compositions. The compositions of the invention can be included as reagents in kits either as starting materials or provided for use in, for example, the methodologies described above.

A kit may include one or more of any of the components useful for practicing the subject methods, as described herein, such as capture agents, supports, reporter complexes, specific binding members, detectable tags, displacement binding members, addressable tags and corresponding array supports, buffers, etc.

In some embodiments, the kit includes: a first capture agent that specifically binds a target analyte; a reporter complex; and a displacement binding member. In certain embodiments of the kit, the reporter complex includes: a first specific binding member linked to a second capture agent that specifically binds the target analyte; and a second specific binding member complementary to the first specific binding member and the first and second specific binding members are specifically bound to form the reporter complex, where the displacement binding member is complementary to the first or second specific binding members. In certain embodiments of the kit, the second specific binding member is linked to one or more detectable tags. In certain embodiments of the kit, the first specific binding member is linked to one or more detectable tags. In certain embodiments of the kit, the second specific binding member comprises a detectable tag. In certain embodiments of the kit, the displacement binding member is complementary to the first specific binding member. In certain embodiments of the kit, the displacement binding member is complementary to the second specific binding member. In certain embodiments of the kit, the first capture agent is linked to a support (e.g., a bead, a particle (e.g., a magnetic particle), a gel, a membrane, a fiber, a biosensor chip surface, a vessel (e.g., a tube surface), cell, or a bacterium). In certain embodiments of the kit, the target analyte is a nucleic acid, a protein, a hormone, a lipid, a small molecule, or a sugar. In certain embodiments of the kit, the first capture agent and the second capture agent are independently selected from a nucleic acid, a protein, a peptide, or a small molecule (e.g., an antibody, a hapten, an aptamer, etc).

The one or more components of the kit may be provided in separate containers (e.g., separate tubes, bottles, or wells in a multi-well strip or plate). The compositions of the kit may be provided in a liquid composition, such as any suitable buffer. Alternatively, the composition may be provided in a dry composition (e.g., a lyophilized, dry powder), and the kit may optionally include one or more buffers for reconstituting the dry composition.

In addition, one or more components of the kit may be combined into a single container, e.g., a glass or plastic vial, tube or bottle. In certain instances, the kit may further include a container (e.g., such as a box, a bag, an insulated container, a bottle, tube, etc.) in which all of the components (and their separate containers) are present. The kit may further include packaging that is separate from or attached to the kit container and upon which is printed information about the kit, the components of the and/or instructions for use of the kit.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, DVD, portable flash drive, etc., on which the information has been recorded. Yet another means that may be present is a website address which may be used via the Internet to access the information at a removed site. Any convenient means may be present in the kits.

### Utility

The compositions, system and methods as described herein may find use in a variety of applications, including diagnostic and research applications, in which the separation, detection and/or analysis of an analyte of interest is desirable.

Such applications include methodologies such as cytometry, microscopy, immunoassays (e.g. competitive or non-competitive), assessment of a free analyte, assessment of receptor bound ligand, detection of biomarkers, and so forth. The compositions, system and methods described herein may be useful in analysis of any of a number of samples, including but not limited to biological fluids, cell culture samples, and tissue samples.

In some instances, the methods and compositions find use the detection of analytes such as toxins, microorganisms, and other disease biomarkers at very low concentrations. Their early detection affords early diagnosis and can greatly increase the success rate of medical treatments. In particular, disease biomarkers such as proteins and nucleic acids may be detected in readily available clinical samples such as plasma, serum, urine, and saliva according to the subject methods.

In some cases, the methods and compositions find use in any assay format where the separation, detection and/or analysis of a target from a sample is of interest, including but not limited to, flow cytometry, in-situ hybridization, enzyme-linked immunosorbent assays (ELISAs), western blot analysis, and magnetic cell separation assays. The subject compositions may be adapted for use in any convenient applications where target analytes are detected in a sample.

### Exemplary Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-87 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below.

1. A method of detecting a target analyte in a sample, comprising:

(a) contacting the sample with:
(i) a first capture agent that specifically binds a target analyte; and
(ii) a reporter complex, comprising:
   (A) a first specific binding member linked to a second capture agent that specifically binds the target analyte;
   (B) a second specific binding member complementary to the first specific binding member, wherein the first and second specific binding members specifically bind to each other to form the reporter complex;

under conditions sufficient to specifically bind the first and second capture agents to the target analyte to produce a sandwich complex;

(b) separating the sandwich complex from the sample; and

(c) releasing the second specific binding member from the sandwich complex using a displacement binding member that is complementary to one of the target analyte, the first capture agent, the second capture agent, the first specific binding member, and the second specific binding member.

2. The method of 1, wherein the second specific binding member is linked to one or more detectable tags.
3. The method of 1, wherein the second specific binding member comprises a detectable tag or a plurality of detectable tags.
4. The method of any one of 2 and 3, further comprising detecting the detectable tag.
5. The method of any one of 1-4, wherein the displacement binding member is complementary to the first specific binding member and step (c) comprises specifically binding the displacement binding member and the first specific binding member.
6. The method of any one of 1-4, wherein the displacement binding member is complementary to the second specific binding member and step (c) comprises specifically binding the displacement binding member and the second specific binding member.
7. The method of any one of 1-6, wherein in step (c), the second specific binding member is released into a solution having a volume that is 50% or less the volume of the sample.
8. The method of any one of 1-7, wherein the target analyte is a nucleic acid, a protein, a hormone, a small molecule, a metabolite, a cell, a bacterium, a virus, a lipid, a biomarker, or a sugar.
9. The method of any one of 1-7, wherein the first capture agent and the second capture agent are independently selected from a nucleic acid, a protein, a peptide, or a small molecule.
10. The method of any one of 1-9, wherein the first capture agent is linked to a support selected from a bead, a particle, a gel, a membrane, a fiber, a biosensor chip surface, a vessel, a cell, a viral particle, a bacteriophage, or a bacterium.
11. The method of any one of 1-10, wherein the first specific binding member, the second specific binding member and the displacement binding member are independently selected from a nucleic acid, a protein, a peptide, a small molecule, or an analog thereof.
12. The method of 11, wherein the first specific binding member, the second specific binding member and the displacement binding member each comprise a nucleic acid.
13. The method of any one of 4-12, wherein the detectable tag comprises an enzyme, a nucleic acid, a polypeptide, a particle, an affinity tag, a fluorophore, a chromophore, a luminescent tag, a radioactive tag or a chemoselective tag.
14. The method of 13, wherein detecting the detectable tag comprises: identifying a nucleic acid or polypeptide, detecting a fluorescent luminescent or radioactive signal, detecting the product of an enzyme-catalyzed reaction, detecting the presence of particles, or chemoselectively attaching a fluorophore to a chemoselective tag.
15. The method of any one of 1-14, wherein:

the reporter complex comprises two or more second specific binding members specifically bound to two or more non-linked first specific binding members; and

step (c) comprises releasing the two or more second specific binding members.

16. The method of 15, wherein each of the two or more second specific binding members is linked to one or more detectable tags.
17. The method of any one of 1-16, wherein the reporter complex is described by the following formula

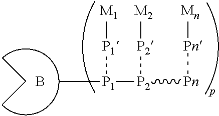

wherein B is the second capture agent;

P₁ to Pₙ are the two or more first specific binding members linked to the second capture agent B, wherein each first specific binding member may be the same or different;

P₁′ to Pₙ′ are the two or more second specific binding members specifically bound to the complementary first binding member P₁ to Pₙ, wherein each second specific binding member may be the same or different;

each M₁ to Mₙ is independently a detectable tag;

each n is independently 2 to 100; and

p is 1 to 100.

18. The method of any one of 1-16, wherein the reporter complex is described by the following formula:

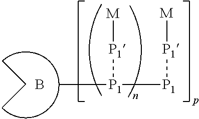

wherein B is the second capture agent;

P₁ is the first specific binding member linked to the second capture agent B;

P₁′ is the second specific binding member specifically bound to the complementary first binding member P₁;

each M is a detectable tag;

each n is independently 0 to 100; and

p is 1 to 100.

19. The method of 18, wherein:

the reporter complex comprises two or more second specific binding members that are complementary to two or more sites of the first specific binding member, wherein each of the two or more second specific binding member is optionally linked to one or more detectable tags; and

step (c) comprises releasing the two or more second specific binding members.

20. The method of 19, wherein each of the two or more second specific binding members is the same.
21. The method of 19, wherein each of the two or more second specific binding members is different.
22. The method of 19, further comprising detecting the one or more detectable tags of the displaced two or more second specific binding members.
23. The method of 1, wherein:

the sample comprises two or more target analytes;

step (a) comprises contacting the sample with two or more distinct reporter complexes each further comprising a distinct addressable tag to produce two or more distinct sandwich complexes; and

step (c) comprises releasing second specific binding members from the two or more distinct sandwich complexes using at least one displacement binding member, wherein the second specific binding members are linked to the distinct addressable tags;

the method further comprising:

(d) capturing the distinct addressable tags on a support.

24. The method of 23, wherein each reporter complex comprises a distinct second specific binding member and step (c) comprises releasing the distinct second specific binding members using two or more distinct displacement binding members.
25. The method of 23, wherein each reporter complex comprises a second specific binding member complementary to the same first specific binding member and step (c) comprises releasing all of the second specific binding members using a single displacement binding member.
26. The method of any one of 24 and 25, wherein the displacement binding member is selected from polypeptides, nucleic acids and small molecules.
27. The method of 26, wherein the displacement binding member is a nucleic acid.
28. The method of 1, wherein the sample comprises two or more target analytes, wherein:

step (a) comprises contacting the sample with:
two or more distinct first capture agents that specifically bind different target analytes; and
two or more distinct reporter complexes, each comprising a distinct second capture agent that specifically bind different target analytes;

wherein the two or more distinct reporter complexes each further comprise a distinct addressable tag and a detectable tag linked to the second specific binding member;

under conditions sufficient to specifically bind each of the two or more distinct first capture agents and the corresponding two or more distinct reporter complexes to different target analytes to produce two or more distinct sandwich complexes;

step (b) comprises separating the two or more distinct sandwich complexes from the sample; and

step (c) comprises releasing the second specific binding members from the two or more distinct sandwich complexes using at least one displacement binding member;

the method further comprising:
(d) capturing two or more distinct addressable tags on a support.
   29. The method of 28, wherein each two or more distinct reporter complexes comprises a distinct second specific binding member and step (c) comprises releasing the distinct second specific binding members using two or more distinct displacement binding members.
   30. The method of 29, wherein the two or more distinct displacement binding members are independently selected from polypeptides, nucleic acids and small molecules.
   31. The method of 30, wherein the two or more distinct displacement binding members are nucleic acids.
   32. The method of 28, wherein each two or more distinct reporter complexes comprises a second specific binding member complementary to the same first specific binding member and step (c) comprises releasing all of the second specific binding members using a single displacement binding member.
   33. The method of 32, wherein the displacement binding member is a nucleic acid.
   34. A composition, comprising:

(a) a first capture agent that specifically binds a target analyte; and

(b) a reporter complex, comprising:
a first specific binding member linked to a second capture agent that specifically binds the target analyte; and
a second specific binding member complementary to the first specific binding member, wherein the first and second specific binding members are specifically bound to form the reporter complex.
   35. The composition of 34, wherein the second specific binding member is linked to one or more detectable tags.
   36. The composition of 34, wherein the second specific binding member comprises a detectable tag or a plurality of detectable tags.
   37. The composition of any one of 34-36, wherein the composition further comprises the target analyte.
   38. The composition of any one of 34-37, wherein the second specific binding member is further linked to an addressable tag.
   39. The composition of any one of 34-37, wherein the reporter complex comprises two or more second specific binding members that are specifically bound to two or more complementary sites of the first specific binding member, wherein each of the two or more second specific binding members is linked to one or more detectable tags.
   40. The composition of any one of 34-39, wherein the first capture agent is linked to a support.
   41. The composition of 34, comprising a displacement binding member that is complementary to one of the target analyte, the first capture agent, the second capture agent, the first specific binding member, and the second specific binding member.
   42. A composition, comprising:

(a) a reporter complex, comprising:
a first specific binding member linked to a first capture agent that specifically binds a target analyte; and
a second specific binding member complementary to the first specific binding member, wherein the first and second specific binding members are specifically bound to form the reporter complex; and

(b) a displacement binding member that is complementary to one of the target analyte, the first capture agent, the first specific binding member, and the second specific binding member.

43. The composition of 42, wherein the second specific binding member is linked to one or more detectable tags.
44. The composition of 42, wherein the second specific binding member comprises a detectable tag or a plurality of detectable tags.
45. The composition of 42, wherein the displacement binding member is complementary to the first specific binding member.
46. The composition of 42, wherein the displacement binding member is complementary to the second specific binding member.
47. The composition of any one of 42-46, further comprising a second capture agent that specifically binds the target analyte.
48. The composition of any one of 46-47, wherein the second capture agent is linked to a support.
49. The composition of any one of 42-48, wherein the composition further comprises the target analyte.
50. The composition of any one of 42-48, wherein the second specific binding member is further linked to an addressable tag.
51. The composition of any one of 42-50, wherein the reporter complex comprises two or more second specific binding members that are specifically bound to two or more complementary sites of the first specific binding member, wherein each of the two or more second specific binding members is linked to one or more detectable tags.
52. A system comprising:

an electrophoresis device comprising an analyte capture zone that comprises;
a first capture agent that specifically binds either directly or indirectly to a target analyte; and
a reporter complex, comprising:
   a first specific binding member linked to a second capture agent that specifically binds the target analyte; and
   a second specific binding member complementary to the first specific binding member, wherein the first and second specific binding members are specifically bound to form the reporter complex.
      53. The system of 52, wherein the second specific binding member is linked to one or more detectable tags.
      54. The system of 52, wherein the second specific binding member comprises a detectable tag or a plurality of detectable tags.
      55. The system of any one of 52-54, wherein the analyte capture zone further comprises a displacement binding member that is complementary to one of the target analyte, the first capture agent, the second capture agent, the first specific binding member, and the second specific binding member.
      56. The system of any one of 52-55, wherein the electrophoresis device further comprises an analyte concentration zone.
      57. A method of detecting a target analyte in a sample, comprising:

(a) contacting the sample with:
(i) a first capture agent that specifically binds a target analyte; and
(ii) a reporter complex, comprising:
   (A) a first specific binding member linked to a second capture agent that specifically binds the target analyte;
   (B) a second specific binding member linked to a detectable tag, wherein the second specific binding member is complementary to the first specific binding member and the first and second specific binding members are specifically bound to form the reporter complex;

under conditions sufficient to specifically bind the first and second capture agents to the target analyte to produce a sandwich complex;

(b) separating the sandwich complex from the sample; and

(c) cleaving the detectable tag from the sandwich complex.

58. The method of 57, wherein step (c) comprises contacting the sandwich complex with an enzyme under conditions sufficient to cleave a portion of the reporter complex that links the detectable tag to the second capture agent.
59. The method of 57, wherein the reporter complex further comprises a cleavable linker that links the detectable tag and the second capture agent and step (c) comprises applying a stimulus to cleave the cleavable linker and release the detectable tag.
60. The method of any one of 57-59, further comprising detecting the detectable tag.
61. The method of any one of 57-60, wherein in step (c), the detectable tag is released into a solution having a volume that is 50% or less the volume of the sample.
62. The method of any one of 57-61, wherein the target analyte is a nucleic acid, a protein, a hormone, a lipid or a sugar.
63. The method of any one of 57-61, wherein the first capture agent and the second capture agent are independently selected from a nucleic acid, a protein, a peptide, or a small molecule.
64. The method of any one of 57-63, wherein the first capture agent is linked to a support.
65. The method of 63 or 64, wherein the target analyte is a target protein and the first capture agent and the second capture agent are each an antibody, an antibody fragment or a derivatized antibody.
66. The method of any one of 57-65, wherein the first specific binding member and the second specific binding member are independently selected from a nucleic acid or nucleic acid analog.
67. The method of any one of 57-66, wherein the detectable tag comprises an enzyme, a nucleic acid, a polypeptide, a particle, an affinity tag, a fluorophore, a chromophore, a luminescent tag, a radioactive tag or a chemoselective tag.
68. A method of detecting a target analyte in a sample, comprising:

(a) contacting the sample with:
(i) a first capture agent that specifically binds a target analyte; and
(ii) a reporter complex, comprising:
   (A) a first specific binding member linked to a second capture agent that specifically binds the target analyte;
   (B) a second specific binding member complementary to the first specific binding member, wherein the first and second specific binding members specifically bind to each other to form the reporter complex;

under conditions sufficient to specifically bind the first and second capture agents to the target analyte to produce a sandwich complex;

(b) separating the sandwich complex from the sample; and

(c) releasing the second specific binding member from the sandwich complex using a displacement binding member.

69. The method of 68, wherein the displacement binding member is complementary to the first or second capture agent.
70. The method of 68, wherein the displacement binding member is complementary to the first or second specific binding members.
71. The method of 68, wherein the displacement binding member is complementary to the target analyte.
72. A kit, comprising:

a first capture agent that specifically binds a target analyte;

a reporter complex, comprising:
a first specific binding member linked to a second capture agent that specifically binds the target analyte; and
a second specific binding member complementary to the first specific binding member, wherein the first and second specific binding members are specifically bound to form the reporter complex; and

a displacement binding member that is complementary to one of the target analyte, the first capture agent, the second capture agent, the first specific binding member, and the second specific binding member.

73. The kit of 72, wherein the second specific binding member is linked to one or more detectable tags.
74. The kit of 72, wherein the second specific binding member comprises a detectable tag or a plurality of detectable tags.
75. The kit of any one of 72-74, wherein the displacement binding member is complementary to the first specific binding member.
76. The kit of any one of 72-74, wherein the displacement binding member is complementary to the second specific binding member.
77. The kit of any one of 72-76, wherein the first capture agent is linked to a support.
78. The kit of any one of 72-77, wherein the target analyte is a nucleic acid, a protein, a hormone, a lipid or a sugar.
79. The kit of any one of 72-77, wherein the first capture agent and the second capture agent are independently selected from a nucleic acid, a protein, a peptide, or a small molecule.
80. The method of any one of 1-33, wherein one or more of the first capture agent, the first specific binding member, the second capture agent, and the second specific binding member are added independently to the sample to form the sandwich complex comprising the reporter complex.
81. The method of any one of 2-33, wherein one or more of the first capture agent, the first specific binding member, the second capture agent, the second specific binding member, and the detectable tag or one or more of the plurality of detectable tags are added independently to the sample to form the sandwich complex comprising the reporter complex.
82. The method of any one of 1-33, wherein the displacement binding member is complementary to the first or second specific binding member.
83. The composition of any one of 34-41, comprising a displacement binding member that is complementary to the first or second binding member.
84. The composition of any one of 42-51, comprising a displacement binding member that is complementary to the first or second binding member.
85. The kit of any one of 72-79, wherein the displacement binding member is complementary to the first or second binding member.
86. The method of any one of 1-33, wherein one or more of the sample, the first capture agent, the first specific binding member, the second capture agent, and the second specific binding member are added independently to the sample to form the sandwich complex comprising the reporter complex.
87. The method of any one of 1-34, wherein one or more of the sample, the first capture agent, the first specific binding member, the second capture agent, the second specific binding member, and the detectable tag or one or more of the plurality of detectable tags are added independently to the sample to form the sandwich complex comprising the reporter complex.

### EXAMPLES

### Example 1

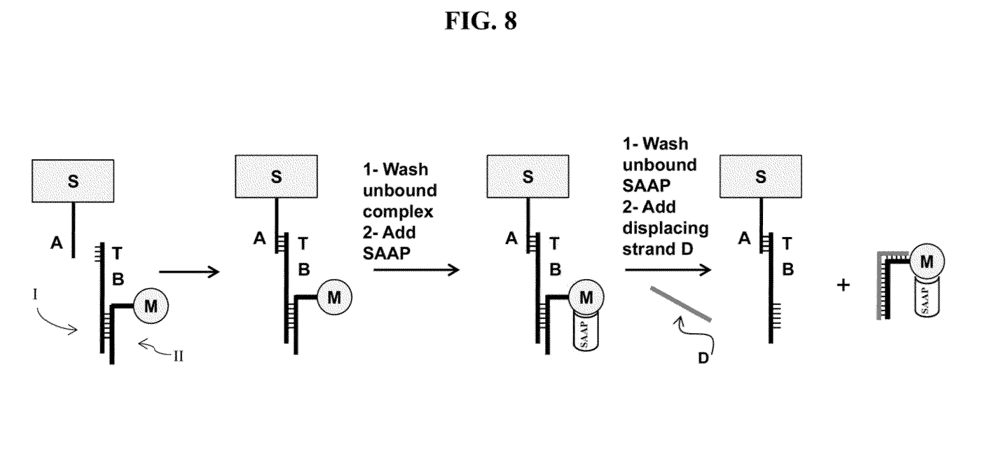

FIG. 8 depicts the assay performed in Example 1. Affinity agent A is a 55-mer oligonucleotide, tethered to a magnetic bead S. The target T and affinity agent B, which binds the target T, are also oligonucleotides and, for the purpose of this example, are fused together to represent the binding of B to T. B is further fused to strand I. Thus T, B, and I are fused into a single oligonucleotide 53-bases long. Target T is captured by affinity agent A by means of a 24 bp sequence complementarity with T. Strand II is a 50-mer oligonucleotide which has biotin linked to one end representing the measurable moiety M. Strand II is bound to strand I through a 27 bp complementary sequence. Displacing strand D is an oligonucleotide 50-mer complementary to the full length of strand II.

In this example, a complex including strand II and strand I with fused B and T, shown in FIG. 8, was captured from 25 μL or 250 μL samples containing varying concentrations of complex in HCB buffer using 2.7 micron magnetic beads modified with oligonucleotide A.

The final concentration of beads in the mix was 4.5 mg/ml. The mix was incubated for 24 hrs. in a rotator. Subsequently, magnetic beads with bound complex were separated from the aqueous phase with a magnet and washed twice with 100 μL or 500 μL of HCB buffer with 100 μg/mL BSA to remove unbound complex. Beads were then suspended in 25 or 250 μL of a streptavidin-alkaline phosphatase conjugate (SAAP), which binds to the biotin tags (M), at 2 μg/mL in the same buffer and incubated in a rotator for 30 minutes. Beads were then separated and unbound SAAP washed away with 100 μL or 500 μL of DB buffer three times. The beads with bound SAAP-labeled complex were suspended in 10 μL of DB supplemented with 1 μM displacing oligonucleotide D and incubated at RT for 30 minutes to displace strand II with bound SAAP and release it into the aqueous phase. Finally, the beads and aqueous phase were separated and 5 μL of the aqueous phase were tested for alkaline phosphatase activity from the displaced SAAP-bound strands in a 25 μL colorimetric assay.

All of the oligonucleotides utilized in this example were synthetic DNA. Conventional methods were used for tethering oligonucleotides to 2.7 micron carboxylated magnetic beads, linkage of biotin moieties to DNA, and colorimetric alkaline phosphatase assay using PNPP substrate. The HCB buffer contains: 50 mM Tris, pH7.5, 0.5M NaCl, 0.1% Tween 20. DB buffer contains: 5 mM Tris pH8, 0.5M NaCl, 0.1% T20, 100 ug/ml BSA. All steps were performed at room temperature.

In this example, the lowest concentration of the target analyte detectable from the 25 μL and 250 μL samples was compared. The colorimetric assay is configured to accept up to a 5 μl sample volume. In this assay, the limit of detection (LOD) when beads are suspended in the original sample volume is estimated at 20 pM. In order to enable measurement of complex concentrations below the LOD of the assay, the complex present in larger 25 μL or 250 μL samples was captured with modified magnetic beads and an SAAP-labeled strand was released by strand displacement into a 10 μL final volume, 5 μL of which were tested for alkaline phosphatase activity in the colorimetric assay.

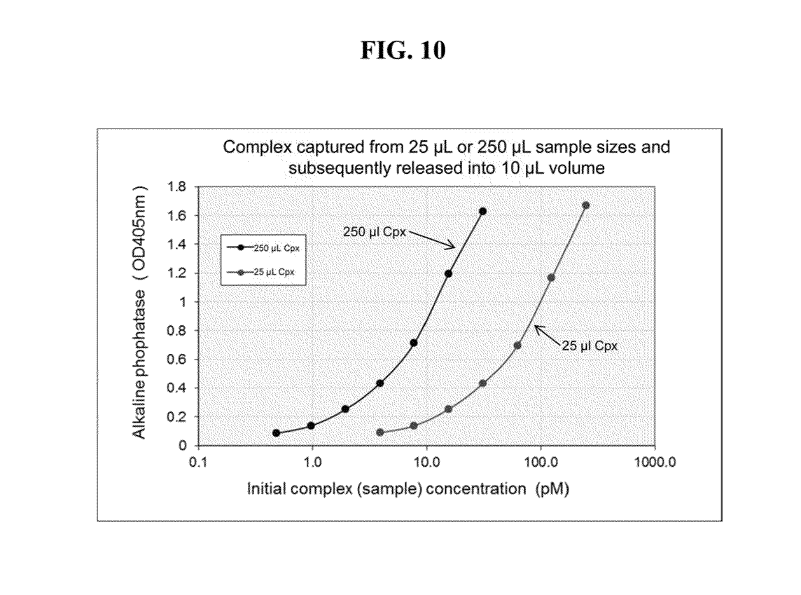

FIG. 10 shows results of the assay demonstrating that the LOD is around 8 pM when the initial sample is 25 μL and the LOD is around 1 pM when the initial sample is 250 μl. Analysis of the remaining beads revealed that displacement of the SAAP labelled strand from the beads was 90-100% (not shown). This example demonstrates how strand displacement methods described herein can be effectively used to prepare a large sample to provide for its quantitation with an assay that only takes a limited sample volume. Furthermore, the entire procedure can be performed under conditions that are compatible with the preservation of the integrity and biological activity of analyte and complex components.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method of detecting a target analyte in a sample, comprising:
(a) contacting the sample with:
(i) a first capture agent that specifically binds a target analyte; and
(ii) a reporter complex, comprising:
(A) a first specific binding member linked to a second capture agent that specifically binds the target analyte;
(B) a second specific binding member complementary to the first specific binding member, wherein the first and second specific binding members specifically bind to each other to form the reporter complex;
under conditions sufficient to specifically bind the first and second capture agents to the target analyte to produce a sandwich complex;
(b) separating the sandwich complex from the sample; and
(c) releasing the second specific binding member from the sandwich complex using a displacement binding member that is complementary to one of the target analyte, the first capture agent, the second capture agent, the first specific binding member, and the second specific binding member.

2. (canceled)

3. The method of claim 1, wherein the second specific binding member comprises a detectable tag or a plurality of detectable tags.

4. The method of claim 3, further comprising detecting the detectable tag.

5. The method of claim 1, wherein the displacement binding member is complementary to the first specific binding member and step (c) comprises specifically binding the displacement binding member and the first specific binding member.

6. The method of claim 1, wherein the displacement binding member is complementary to the second specific binding member and step (c) comprises specifically binding the displacement binding member and the second specific binding member.

7. The method of claim 1, wherein in step (c), the second specific binding member is released into a solution having a volume that is 50% or less the volume of the sample.

8.-9. (canceled)

10. The method of claim 1, wherein the first capture agent is linked to a support selected from a bead, a particle, a gel, a membrane, a fiber, a biosensor chip surface, a vessel, a cell, a viral particle, a bacteriophage, or a bacterium.

11. (canceled)

12. The method of claim 1, wherein the first specific binding member, the second specific binding member and the displacement binding member each comprise a nucleic acid.

13.-14. (canceled)

15. The method of claim 1, wherein:
the reporter complex comprises two or more second specific binding members specifically bound to two or more non-linked first specific binding members; and
step (c) comprises releasing the two or more second specific binding members.

16. The method of claim 15, wherein each of the two or more second specific binding members is linked to one or more detectable tags.

17. The method of claim 1, wherein the reporter complex is described by the following formula 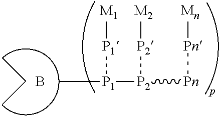
wherein B is the second capture agent;
P₁ to Pₙ are the two or more first specific binding members linked to the second capture agent B, wherein each first specific binding member may be the same or different;
P₁′ to Pₙ′ are the two or more second specific binding members specifically bound to the complementary first binding member P₁ to Pₙ, wherein each second specific binding member may be the same or different;
each M₁ to Mₙ is independently a detectable tag;
each n is independently 2 to 100; and
p is 1 to 100.

18. The method of claim 1, wherein the reporter complex is described by the following formula: 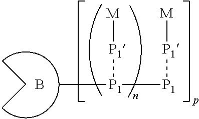
wherein B is the second capture agent;
P₁ is the first specific binding member linked to the second capture agent B;
P₁′ is the second specific binding member specifically bound to the complementary first binding member P₁;
each M is a detectable tag;
each n is independently 0 to 100; and
p is 1 to 100.

19. The method of claim 18, wherein:
the reporter complex comprises two or more second specific binding members that are complementary to two or more sites of the first specific binding member, wherein each of the two or more second specific binding member is optionally linked to one or more detectable tags; and
step (c) comprises releasing the two or more second specific binding members.

20. The method of claim 19, wherein each of the two or more second specific binding members is the same.

21. The method of claim 19, wherein each of the two or more second specific binding members is different.

22. The method of claim 19, further comprising detecting the one or more detectable tags of the displaced two or more second specific binding members.

23. The method of claim 1, wherein:
the sample comprises two or more target analytes;
step (a) comprises contacting the sample with two or more distinct reporter complexes each further comprising a distinct addressable tag to produce two or more distinct sandwich complexes; and
step (c) comprises releasing second specific binding members from the two or more distinct sandwich complexes using at least one displacement binding member, wherein the second specific binding members are linked to the distinct addressable tags;
the method further comprising:
(d) capturing the distinct addressable tags on a support.

24. The method of claim 23, wherein each reporter complex comprises a distinct second specific binding member and step (c) comprises releasing the distinct second specific binding members using two or more distinct displacement binding members.

25. The method of claim 23, wherein each reporter complex comprises a second specific binding member complementary to the same first specific binding member and step (c) comprises releasing all of the second specific binding members using a single displacement binding member.

26.-33. (canceled)

34. A composition, comprising:
(a) a first capture agent that specifically binds a target analyte; and
(b) a reporter complex, comprising:
a first specific binding member linked to a second capture agent that specifically binds the target analyte; and
a second specific binding member complementary to the first specific binding member, wherein the first and second specific binding members are specifically bound to form the reporter complex.

35.-41. (canceled)

42. A composition, comprising:
(a) a reporter complex, comprising:
a first specific binding member linked to a first capture agent that specifically binds a target analyte; and
a second specific binding member complementary to the first specific binding member, wherein the first and second specific binding members are specifically bound to form the reporter complex; and
(b) a displacement binding member that is complementary to one of the target analyte, the first capture agent, the first specific binding member, and the second specific binding member.

43.-51. (canceled)

52. A system comprising:
an electrophoresis device comprising an analyte capture zone that comprises;
a first capture agent that specifically binds either directly or indirectly to a target analyte; and
a reporter complex, comprising:
a first specific binding member linked to a second capture agent that specifically binds the target analyte; and
a second specific binding member complementary to the first specific binding member, wherein the first and second specific binding members are specifically bound to form the reporter complex.

53.-71. (canceled)

72. A kit, comprising:
a first capture agent that specifically binds a target analyte;
a reporter complex, comprising:
a first specific binding member linked to a second capture agent that specifically binds the target analyte; and
a second specific binding member complementary to the first specific binding member, wherein the first and second specific binding members are specifically bound to form the reporter complex; and
a displacement binding member that is complementary to one of the target analyte, the first capture agent, the second capture agent, the first specific binding member, and the second specific binding member.

73.-87. (canceled)
